(19) United States
(12) Patent Application Publication
(10) Pub. No.: US 2015/0366943 A1
(43) Pub. Date: Dec. 24, 2015

(54) MOBILIZATION OF HEMATOPOIETIC STEM CELLS FROM BONE MARROW TO BLOOD USING A COMBINATION OF A ROBO4 RECEPTOR ANTAGONIST AND A CXCR4 ANTAGONIST OR hrVEGF-165 AND A CXCR4 ANTAGONIST

(71) Applicant: The Regents of the University of California, Oakland (CA)

(72) Inventors: E. Camilla Forsberg, Santa Cruz, CA (US); Dr. Stephanie Smith-Berdan, Santa Cruz, CA (US)

(73) Assignee: The Regents of the University of California, Oakland (CA)

(21) Appl. No.: 13/261,991

(22) PCT Filed: May 17, 2013
(86) PCT No.: PCT/US2013/041724

§ 371 Date: Nov. 17, 2014

Related U.S. Application Data
(60) Provisional application No. 61/648,174, filed on May 17, 2012.
(60) Provisional application No. 61/769,478, filed on Feb. 26, 2013.

(51) Int. Cl. A61K 38/19; A61K 38/18; A61K 38/17

(52) U.S. Cl. CPC A61K 38/193; A61K 38/17; A61K 38/1866

(57) ABSTRACT

The invention herein disclosed provides for compositions, methods for synthesizing said compositions, and methods for using said compositions, wherein the compositions and methods may be used in clinical treatment using hematopoietic transplantation, particularly to those relating to treatment of cancer and metastasis. The compositions regulate mobilization of hematopoietic stem cells (HSCs) between bone marrow (BM) and the peripheral blood (PB).

### RELATIONSHIP TO OTHER APPLICATIONS

This application claims priority to and benefits of the following: U.S. Provisional Patent Application No. U.S. 61/648,174 filed 17 May 2012, entitled “Improved Mobilization Of Hemaopoetic Stem Cells From Bone Marrow To Blood Using A Combination Of A Robo4 Receptor Antagonist And A Cxcr4 Antagonist” and U.S. Provisional Patent Application No. U.S. 61/769,478 filed 26 Feb. 2013, entitled “Mobilization Of Hemaopoetic Stem Cells From Bone Marrow To Blood Using A Combination Of AMD3100 and rhVEGF-165”, which are both herein incorporated by reference in its entirety for all purposes.

### FIELD OF THE INVENTION

The present invention relates to using compositions that regulate mobilization of hematopoietic stem cells (HSCs) between bone marrow (BM) and the blood.

### BACKGROUND

The tremendous potential of stem cells to provide a complete and permanent cure for a wide range of human disorders makes progress in improving the safety and efficiency of cell-based therapies a top priority in modern medicine. Successful hematopoietic cell transplantations have been performed for over 50 years and have made HSC the paradigm for stem cell therapy. Still, the morbidity and mortality of hematopoietic transplant recipients are unacceptably high and transplants are therefore reserved for patients with few other treatment options. By investigating the molecular mechanisms of HSC interaction with the bone marrow (BM) microenvironment, our goal is to enable specific and efficient manipulation of both HSC mobilization and engraftment.

Because mobilized peripheral blood (PB) is an increasingly common source of HSC, transplantation therapy involves HSC movement both into and out of the BM. In mice, as well as in humans, combined administration of cytoxan (cyclophosphamide) and G-CSF (Cy/G treatment) induces self-renewing divisions of BM HSC, resulting in an expansion of the HSC pool followed by migration of HSC to the blood stream (Morrison et al., 1997; Passegue et al., 2005; Wright et al., 2001). More recently, AMD3100, an antagonist of the G protein-coupled receptor Cxcr4, has been used to mobilize hematopoietic cells (Broxmeyer et al., 2005; Liles et al., 2003; Watt and Forde, 2008). The mechanisms of Cy/G and AMD3100-induced mobilization are fundamentally different. HSC mobilization with Cy/G requires daily injections of G over several days and acts by indirect mechanisms, as HSC do not express the G-CSF receptor (Liu et al., 2000). HSC expand several fold in the first days of treatment, followed by relocation to the blood stream. In contrast, AMD3100-induced mobilization is rapid, with increased numbers of progenitors detected in the blood one hour after administration of a single dose of drug, and thus does not involve cell expansion. Upon transplantation, intravenously injected HSC must find their way back to the BM and engraft. Most likely, HSC home in response to chemokines, including the Cxcr4 ligand Sdf1 (also known as Cxcl12), followed by adhesion to the niche by engaging in specific interactions with cellular and matrix components. Engraftment of transplanted HSC requires that HSC-supportive niches in the host are vacated by resident HSC to allow donor HSC access to these sites. Partial or complete myeloablation, by cytotoxic drugs and/or irradiation, is currently necessary to accomplish this. The ability to long-term engraft is a defining and unique property of HSC and critically important for both normal hematopoietic development and transplantation therapy.

Sdf1 and Cxcr4 play pivotal roles in HSC location and function. Mice deficient in either Sdf1 or Cxcr4 die during late embryogenesis and lack BM hematopoiesis (Nagasawa et al., 1996; Zou et al., 1998). As described above, the Cxcr4 antagonist AMD3100 can be used to mobilize hematopoietic progenitors from the BM to PB in mice and humans (Broxmeyer et al., 2005; Watt and Forde, 2008), and Cxcr4 blocking antibodies impair HSC engraftment (Peled et al., 1999). In addition, HSC actively migrate toward Sdf1 in transwell migration assays (Lapidot, 2001; Wright et al., 2002), and recent data suggest that HSC specifically localize next to BM cells expressing high levels of Sdf1 (Sugiyama et al., 2006). Thus, there is extensive evidence supporting critical roles for Sdf1 and Cxcr4 in regulating HSC location.

Surprisingly, however, deletion of Cxcr4 in adulthood results in HSC capable of homing and engraftment (Nie et al., 2008; Sugiyama et al., 2006). In addition, many cells other than HSC express Cxcr4, making it unlikely that Cxcr4, alone, specifies HSC location to stem cell supportive niches. In search for HSC-specific receptors capable of specifying cell location, we recently identified the single-transmembrane receptor Robo4 on HSC by gene expression microarray analysis (Forsberg 2005). A subsequent report confirmed that Robo4 marks longterm reconstituting HSC (Shibata et al., 2009). Robo4, like its family members Robot-3, is capable of regulating cell location by responding to the Slit family of secreted ligands (Kaur et al., 2006; Park et al., 2003; Seth et al., 2005; Suchting et al., 2005). Other than HSC, Robo4 expression seems restricted to endothelial cells, where it functions to regulate blood vessel sprouting (Huminiecki et al., 2002; Park et al., 2003). Robo4^{-/-} mice, though grossly normal, have defects in VEGF- and Slit-induced regulation of vascular integrity and angiogenesis (Jones et al., 2008; London et al.; Marlow et al., 2010).

Of particular relevance are U.S. Pat. No. 6,270,995 to Goodman et al. that discloses recombinant polynucleotides encoding a slit polypeptide, U.S. Pat. No. 6,861,228 to Goodman et al., U.S. Pat. No. 6,270,984 to Goodman et al., and U.S. Pat. No. 6,046,015 to Goodman et al., that disclose methods for modulating Robo: ligand interactions. Also of relevance are publication of Bonig, H., Chudziak, D., Priestley, G., and Papayannopoulou, T. (2009). Insights into the biology of mobilized hematopoietic stem/progenitor cells through innovative treatment schedules of the CXCR4 antagonist AMD3100. Exp Hematol 37, 402-415 e401; Broxmeyer, H. E., Orschell, C. M., Clapp, D. W., Hangoc, G., Cooper, S., Plett, P. A., Liles, W. C., Li, X., Graham-Evans, B., Campbell, T. B., Calandra, G., Bridger, G., Dale, D. C., and Srour, E. F. (2005). Rapid mobilization of murine and human hematopoietic stem and progenitor cells with AMD3100, a CXCR4 antagonist. J Exp Med 201, 1307-1318; and Flomenberg, N., DiPersio, J., and Calandra, G. (2005). Role of CXCR4 chemokine receptor blockade using AMD3100 for mobilization of autologous hematopoietic progenitor cells. Acta Haematol 114, 198-205.

There is therefore a need in the art to provide for a hematopoietic stem cell (HSC)-specific adhesion molecule that cooperates with Cxcr4 to localize HSC to bone marrow (BM) niches. It is highly desirable that new compositions and combinations thereof are developed for treatment of diseases, conditions, and disorders where stem cell therapy is of use.

### BRIEF DESCRIPTION OF THE INVENTION

The present invention relates to using compositions that regulate mobilization of hematopoietic stem cells (HSCs) between bone marrow (BM) and the blood.

The invention contemplates using a composition, the composition comprising a Robo4 receptor antagonist and a Cxcr4 antagonist that show an improved rate of successful mobilization of the hematopoietic stem cells between the BM and the blood. Using the combination results in significant decrease in time between treatment and mobilization of the HSCs, the time for successful mobilization being hours rather than days when current methodologies are used.

In another embodiment, the invention contemplates using the combination of a Robo4 receptor antagonist and a Cxcr4 antagonist with the inclusion further of a CSF receptor agonist. In one exemplary embodiment, the Robo4 receptor antagonist is a Slit ligand.

Exemplary Slit ligands and variants thereof are disclosed by Goodman et al. (US Patent Application No. (USPAN) 20050196783 A1, published Sep. 8, 2005; ibid USPAN 20030170727 A1, published Sep. 11, 2003, ibid USPAN 20090155928 A1, published Jun. 18, 2009; Li, et al., USPAN 20060160729 A1, published Jul. 20, 2006; van Zant et al., USPAN 20090028831 A1, published Jan. 29, 2009 and Li, et al., USPAN 20100069301 A1, published Mar. 18, 2010). The Slit ligand or variant may be derived from any animal species or may be a synthetic protein having Robo4 receptor-binding activity. An exemplary Slit ligand is Slit2. An exemplary Slit ligand variant is Slit homologue Slit-like 2 (see, for example, Holmes, G. P. et al. Mech. Dev. 79:57-72 (1998); Piper, M. et al. Mech. Dev. 94: 213-217 (2000)). The invention may also comprise using an antibody having affinity for Robo4, such as disclosed in Koch et al., USPAN 20080247951 A1, published Oct. 9, 2008). The Slit/Robo guidance system is described in Zou, Cell 102: 363-375 (2000).

In another exemplary embodiment, the chemokine receptor Cxcr4 receptor anatagonist is selected from the group consisting of a stromal cell-derived factor (SDF1) ligand and variants thereof, AMD3100, and a bicyclam derivate. For example, SDF1 alpha and 1 beta are small cytokines belonging to the intercrine CXC subfamily (Shirozu et al., Genomics 28:495-500 (1995)). Certain analogues of N-terminal peptides of the chemokine SDF-1 are CXCR4 antagonists (Loetscher et al. J Biol Chem 273:22279-22283 (1998)). In addition, Staudinger et al. have demonstrated that another CXCR4 ligand, the HIV-1 envelope protein gp 120, effectively antagonizes the effect of SDF-1 (Biochem Biophys Res Commun. 280:1003-1007 (2001)). Ligand variants or homologs may serve as antagonists or agonists. CXCR4 activity can also be modulated by interfering with the receptor itself, rather than inhibiting agonist binding. Tarasova et al. demonstrate expression of a peptide derived from the transmembrane domains of CXCR4 inhibits receptor signaling and HIV replication at concentrations as low as 0.2 μM (J Biol Chem 274:34911-34915 (1999)).

In yet another embodiment, the colony-stimulating factor (CSF) receptor agonist is selected from the group consisting of a granulocyte CSF (G-CSF) and variants thereof, and other natural and synthetic CSFs, myelopoietin, and progenipoietin (ProGP). The invention contemplates using a vascular permeability inducing factor (such as recombinant human vascular endothelial growth factor, 165 amino acid soluble isoform [rhVEGF-165]) and a Cxcr4 inhibitor (such as AMD3100) that show an improved rate of successful mobilization of the hematopoietic stem cells between the BM and the blood. Using the combination results in significant decrease in time between treatment and mobilization of the HSCs, the time for successful mobilization being 2 hours or less rather than days when current methodologies are used. In another embodiment the composition comprises biological activity, the biological activity comprising mobilization of the hematopoietic stem cells between the bone marrow (BM) and the blood.

In another embodiment, the invention contemplates using the combination of a Cxcr4 antagonist with the inclusion further of a CSF receptor agonist.

In another embodiment, the chemokine receptor Cxcr4 receptor anatagonist is selected from the group consisting of a stromal cell-derived factor (SDF1) ligand and variants thereof, AMD3100, and a bicyclam derivate. For example, SDF1 alpha and 1 beta are small cytokines belonging to the intercrine CXC subfamily (Shirozu et al., Genomics 28:495-500 (1995)). Certain analogues of N-terminal peptides of the chemokine SDF-1 are CXCR4 antagonists (Loetscher et al. J Biol Chem 273:22279-22283 (1998)). In addition, Staudinger et al. have demonstrated that another CXCR4 ligand, the HIV-1 envelope protein gp 120, effectively antagonizes the effect of SDF-1 (Biochem Biophys Res Commun. 280:1003-1007 (2001)). Ligand variants or homologs may serve as antagonists or agonists. CXCR4 activity can also be modulated by interfering with the receptor itself, rather than inhibiting agonist binding. Tarasova et al. demonstrate expression of a peptide derived from the transmembrane domains of CXCR4 inhibits receptor signaling and HIV replication at concentrations as low as 0.2 uM (J Biol Chem 274:34911-34915 (1999)).

In yet another embodiment, the colony-stimulating factor (CSF) receptor agonist is selected from the group consisting of a granulocyte CSF (G-CSF) and variants thereof, other natural and synthetic CSFs, myelopoietin, and progenipoietin (ProGP).

In another embodiment the composition comprises biological activity, the biological activity comprising mobilization of the hematopoietic stem cells between the bone marrow (BM) and the blood.

In another embodiment the invention contemplates use of a composition comprising a Robo4 receptor antagonist and a Cxcr4 antagonist for the manufacture of a composition for mobilization of hematopoietic stem cells between bone marrow (BM) and blood. In a preferred embodiment the Robo4 receptor antagonist is a slit ligand. In a more preferred embodiment the slit ligand is selected from the group consisting of slit, slit2, and slit-like2. In a preferred embodiment the Cxcr4 antagonist is selected from the group consisting of AMD3100, stromal cell-derived factor (SDF1) ligand, HIV-1 envelope protein gp120, and a bicyclam derivate.

In another embodiment, the use further comprises combining the composition with a CSF receptor agonist. In a more preferred embodiment, the CSF receptor agonist is selected from the group consisting of G-CSF, myelopoietin, and progenipoietin (ProGP).

In an alternative embodiment the invention contemplates use of a composition comprising a vascular permeability inducing factor and a Cxcr4 antagonist for the manufacture of a composition for mobilization of hematopoietic stem cells between bone marrow (BM) and blood. In a preferred embodiment the vascular permeability inducing factor is selected from the group consisting of VEGF-A, VEGF-B, VEGF-C, VEGF-D, VEGF-E, recombinant human vascular endothelial growth factor, rhVEGF-165, placental growth factor-1 (PGF1) and PGF2. In a more preferred embodiment the vascular permeability inducing factor is a recombinant human vascular endothelial growth factor, 165 amino acid soluble isoform (rhVEGF-165). In a more preferred embodiment the Cxcr4 antagonist is selected from the group consisting of AMD3100, a stromal cell-derived factor (SDF1) ligand, HIV-1 envelope protein gp120, and a bicyclam derivate.

In another embodiment, the use further comprises combining the composition with a CSF receptor agonist. In a preferred embodiment, the CSF receptor agonist is selected from the group consisting of G-CSF, myelopoietin, and progenipoietin (ProGP).

The invention also contemplates a method for improving the rate of successful mobilization of hematopoietic stem cells between bone marrow (BM) and blood, the method comprising the steps of (i) providing the composition of claim 1 or 9; (ii) introducing the composition to the vicinity of the bone marrow and hematopoietic stem cells; allowing the composition to exert a biological effect, the method resulting in improving the rate of successful mobilization of hematopoietic stem cells between bone marrow (BM) and blood. In a preferred embodiment the composition is in combination with a pharmaceutically acceptable excipient.

In another embodiment, the invention contemplates methods of making and using the subject compositions in diagnosis, therapy (for example, to modulate growth and differentiation of hematopoietic tissue in vitro and in vivo), and in the biopharmaceutical industry (for example, combining the subject compositions with a pharmaceutically acceptable excipient and/or other suitable composition, to create a pharmaceutical composition for use in the treatment of hematopoietic disorders, and the like.

The disclosed combination of compositions has important implications in clinical treatment using hematopoieic transplantation, particularly to those relating to treatment of cancer and metastasis.

### BRIEF DESCRIPTION OF THE DRAWINGS

FIG. 1 illustrates that Robo4 is selectively expressed by BM-localized hematopoietic stem cells. (A) Relative levels of Robo4 transcripts in purified BM populations by qRT-PCR compared to HSC. Data shown are from four independent experiments with qPCR reactions performed in triplicate. (B) Relative Robo4 mRNA levels by qRT-PCR in wild-type (wt) HSC, mobilized HSC (M-HSC), and leukemic HSC (L-HSC). (C) Quantitative RT-PCR revealed that Robo4^{-/-} expression increases as HSC (defined as cKit⁺/Lin⁻/Sca1⁺ cells) transition from fetal liver to BM during development. (D) Cell surface Robo4 expression on BM subpopulations from wt mice, demonstrating highly selective Robo4 expression on HSC. (E) Flow cytometry plots of cKit⁺/Lin⁻/Sca1⁺ BM cells from wt and Robo4^{-/-} mice demonstrating the specificity of the antibody for Robo4. (F) Robot cell surface protein was readily detectable in wt brain, but not in Robo4^{-/-} brain or in wt BM, PB, or CD4+T cells. BM, bone marrow; PB, peripheral blood. Error bars represent SEM. *p<0.005; **p<0.0001. See FIG. 8 for cell surface phenotypes and flow cytometry profiles.

FIG. 2 illustrates that Robo4^{-/-} HSC displayed impaired BM localization at steady-state and upon transplantation. HSC frequencies were significantly lower in the BM (A) and higher in PB (B) in Robo4^{-/-} mice compared to wt mice. Other cell types were not affected by Robo4 loss. (C) Robo4^{-/-} HSC had drastically impaired long-term reconstitution potential upon transplantation compared to wt HSC. Total donor-derived cells in PB at the indicated timepoints after competitive reconstitution with 100 wt and Robo4^{-/-} HSC are shown. (D) Relative lineage readout was not affected by Robo4 deficiency. The ratios of mature B, T and myeloid cells in PB, BM, and spleen>16 weeks after competitive transplantation of 100 wt and Robo4 HSC are shown. (E) Robo4^{-/-} HSC gave rise to in vivo spleen colonies with normal frequencies. Lethally irradiated mice were transplanted with either 100 Robo4^{-/-} or wt HSC. Twelve days following transplant, spleens were harvested for CFU-S analysis. (F) The numbers of Robo4-1 HSC and progenitor cells in the BM of transplanted mice was significantly lower than wt cells at >16 weeks post-transplantation. All data are from at least three independent experiments with at least three mice per group. Error bars represent SEM. **p<0.004; ***p<0.0006. See also FIG. 9.

FIG. 3 illustrates that Robo4^{-/-} HSC mobilized less efficiently with Cy/G treatment due to upregulation of Cxcr4. (A) Cy/G injection and tissue analysis schedule. (B) HSC expansion in the BM in response to Cy/G was normal in Robo4^{-/-} mice. (C) Fewer Robo4^{-/-} HSC relocated to the PB at day 2 of Cy/G treatment. No differences between wt and Robo4^{-/-} HSC were observed at day 4. (D) The number of MPP mobilized to the blood was not affected by Robo4 deficiency. (E) Cxcr4 mRNA levels were significantly higher in Robo4^{-/-} HSC compared to wt HSC. (F) Robo4^{-/-} HSC HSC displayed higher Cxcr4 cell surface levels than wt USC by flow cytometry analysis. (G) BM stromal (CD45″Ter119″) cells from Robo4^{-/-} mice expressed higher levels of Sdf1 than wt stromal cells. (H) Slit2 mRNA levels in BM stromal cells were not affected by loss of Robo4. Cy/G, cytoxan/G-CSF. Error bars represent SEM. *p<0.05; **p<0.001. See also FIG. 10.

FIG. 4 illustrates that Robo4^{-/-} HSC were more responsive to AMD3100 than were wt HSC. (A) Injection and analysis schedule for panels B and C. PB was analyzed one hour after AMD3100 injections on day 2. (B) Robo4^{-/-} HSC, but not wt HSC, were mobilized more efficiently by Cy/G+AMD3100 than by Cy/G alone. (C) Mobilization of MPP was more efficient when AMD3100 was added to the Cy/G treatment. No differences were observed between wt and Robo4^{-/-} MPP. (D) Hematopoietic progenitors were more efficiently mobilized with AMD3100 compared to HSC. Wt mice were subjected to two AMD3100 injections one hour apart, with PB analysis one hour after the second injection. (E) Robo4^{-/-} HSC were more efficiently mobilized with AMD3100 compared to wt HSC. Injection and analysis schedule as in D. MPP, multipotent progenitors; MyPro, myeloid progenitors (cKit⁺/Lin^{neg}/Sca1⁻ cells). Error bars represent SEM. The data represent at least three independent experiments with at least three mice per cohort per experiment. *p<0.03; **p<0.01.

FIG. 5 illustrates that HSC expressed lower levels of Cxcr4 and migrated less efficiently toward Sdf1 compared to more mature hematopoietic subpopulations. HSC expressed relatively low levels of Cxcr4 by (A) qRT-PCR analysis, and (B, C) flow cytometry cell surface staining. (D) Transwell migration assays revealed that HSC migration efficiency toward Sdf1 was lower than that of cells expressing higher levels of Cxcr4. The data represent at least three independent experiments. Error bars represent SEM. *p<0.03; **p<0.0001; ***p<0.00001. See also FIG. 11.

FIG. 6 illustrates that combined loss of Robo4 and Cxcr4 function impaired HSC localization to the BM after transplantation. (A) Preincubation of cells with increasing amounts of AMD3100 inhibited migration toward Sdf1 in vitro. (B) Fewer HSC localized to the BM three hours after transplantation when Robo4 and/or Cxcr4 function was blocked. CFSE-labeled cells from wt and Robo4^{-/-} mice with and without AMD3100 preincubation were injected IV into lethally irradiated recipients, followed by tissue analysis for CFSE-positive cells three hours later. (C) A reciprocal increase of Robo4^{-/-} and AMD3100-treated HSC was detected in PB three hours after transplantation. (D) No significant differences in spleen localization were detected. Data represent three individual experiments comprised of 3-4 mice per cohort. Error bars represent SEM.*p<0.02; **p<0.003; ***p<0.0001.

FIG. 7 illustrates a simplified model of Robo4- and Cxcr4-mediated control of HSC migration, engraftment, and mobilization. During developmental transition of HSC location from fetal liver to BM, or upon transplantation, HSC home toward BM niches by the attractant cues between Cxcr4 and stromal-derived Sdf1. Adhesive interactions provided by both Cxcr4 and Robo4 promote stable interactions with the niche with long-term engraftment as a result. B cells and other cells expressing high levels of Cxcr4 also home to the BM, but, similar to Robo4^{-/-} HSC, fail to engage in stable niche interactions. AMD3100-induced mobilization of HSC into the blood stream is more efficient when Robo4 is deleted, in spite of increased levels of Cxcr4.

FIG. 8 illustrates antibodies, cell surface phenotypes and flow cytometry gating strategies for identification of hematopoietic stem and progenitor cells.

FIG. 9 shows that Robo4^{-/-} HSC proliferation rates in vivo and in vitro are similar to wt HSC. (A) HSC and MPP frequencies in the spleen were not affected by Robo4 deletion. The frequencies of HSC and MPP in Robo4^{-/-} spleens were plotted relative to wt mice. Error bars represent SEM. (B) Cell cycle profiles were obtained by flow cytometry of DAPI- and antibody-stained BM cells from wt and Robo4^{-/-} mice. (C) Quantitation of data shown in B. The proportion of HSC in cycle was significantly lower compared to other progenitor cells, but no differences were detected between wt and Robo4^{-/-} HSC. (D) HSC proliferation in vitro was not affected by Robo4 loss. HSC were isolated from BM of wt or Robo4^{-/-} mice by FACS and plated at equal cell numbers/well per strain. Cells were counted at the indicted timepoints by addition of FITC labeled counting beads followed by flow cytometry. (E) Addition of Slit2 (4 ug/ml) did not affect the in vitro proliferation rates of wt HSC. Experiments were performed as in D. (F) Hematopoietic recovery upon 5-FU treatment was normal in Robo4^{-/-} mice. Wt and Robo4^{-/-} mice were injected weekly IP with 5-fluorouracil (100 mg/kg or 120 mg/kg) without uncovering significant differences in survival. Data is from at least two independent experiments. Error bars represent SEM. ***p<0.0001.

FIG. 10 show that wildtype (wt) and Robo4^{-/-} HSC have similar levels of cell surface CD31, Vcam1, and Esam1, and of histone H3 trimethylation in the Cxcr4 locus. (A) Cell surface levels of Vcam1, CD31, and Esam1 are equally high on wt and Robo4^{-/-} HSC. BM cell suspensions from wt and Robo4^{-/-} mice were incubated with antibodies recognizing HSC (Figure Si) and Vcam1, CD31, or Esam1 and analyzed by flow cytometry. HSC were defined as cKit⁺/Lin^{neg}/Sca1⁺/Flk2⁺/CD34⁻ (Vcam1 and CD31 analyses) or cKit⁺/Lin^{neg}/Sca1⁺/Flk2⁻/Slamf1⁺ (Esam1 analysis) BM cells. (B) Schematic of the Cxcr4 locus, indicating the location of primers used for chromatin immunoprecipitation (ChIP) analysis. (C) ChIP with antibodies recognizing the histone marks H3K4me3 (active chromatin) and H3K27me3 (silenced chromatin) showed no significant differences in levels of enrichment of Cxcr4 promoter and intronic regions between wt and Robo4^{-/-} KLS (cKit⁺/Lin^{neg}/Sca1⁺) cells. Data represent three independent experiments, error bars indicate Standard Error of the Mean (SEM).

FIG. 11 shows that Slit2 does not affect the migration of HSC, myeloid progenitors, B or T cells in transwell migration assays. (A) Purified Sdf1 (100 ng/ml) with and without Slit2 (250 or 500 ng/ml) was placed in bottom wells and the migration of lineage-depleted BM cells after a 2-hour incubation period were quantitated by flow cytometry. No significant differences in cell migration toward Sdf1 were observed when adding up to 5-fold excess amounts of Slit2. (B) Slit2 had no effect on T cell migration by itself (black bars) or in combination with Sdf1 (purple bars) in vitro. Experiment performed as in A, except unfractionated BM cells were placed in top wells. (C) Slit2 attenuated HL60 cell migration toward fMLP. Data shown are representative of at least two (C) or three (A and B) independent experiments. Error bars represent SEM. *p<0.03.

FIG. 12 illustrates more efficient mobilization of hematopoietic stem cells by combined administration of Slit2 and AMD3100. 8-16 week old C57BIa mice were injected IV with either HBSS (supplemented with the buffer from Slit2N production), AMD3100 (5 mg/kg), Slit2 (5 ug/mouse equates to 0.25 mg/kg), or AMD3100+Slit2 (at doses listed). Mice either received: two doses of HBSS; single dose of HBSS followed by single dose of Slit2 1 hour later; double dose of AMD3100 separated by one hour; or single dose of AMD3100 only followed up by a dose of AMD3100+Slit2 separated by one hour. Mice were allowed to rest for 1 hour, followed by euthanized by CO₂ inhalation. The whole peripheral blood from each mouse was individually collected via whole body perfusion with 20 mM EDTA, followed by excision of femurs and tibias from each mouse. Single cell suspension from the BM was generated by crushing the bones with a mortar and pestle, red blood cells were lysed during a 5-minute incubation in 0.15 M ammonium chloride and 0.01 M potassium bicarbonate solution on ice, and finally the cell suspension was filtered through a 70 micron nylon mesh. RBCs from the peripheral blood aliquots were sedimented using a 2% dextrose solution while cells incubated at 37° C. The remaining WBC suspension was treated with ACK to remove remaining RBC. The single cell suspensions from both tissues were individually stained with the appropriate antibody cocktail solution contingent on the use of the cells. All antibody incubations were performed on ice for 20-45 minutes with an appropriate concentration of antibody. We analyzed the different cell populations using a 3-laser FACSAria (BD Biosciences, San Jose, Calif.). Flowjo Software (Ashland Oreg.) was used to analyze the expression profiles of the various subsets.

FIG. 13 shows engraftment of wt HSC is impaired in Robo4^{-/-} recipients. WBM from wt mice was transplanted into wt or Robo4^{-/-} recipients conditioned with radiation only (is Rad′, left) and radiation in combination with the CXCR4 inhibitor AMD3100 (Rad+A, middle and right). PB (left and middle panels) and BM (right) donor chimerism at >16 wks post-transplantation are shown. Data represent three (PB) or two (BM) independent experiments with several mice per experiment and group. Four additional experiments under slightly different conditions (cell numbers transplanted or extent of host conditioning) resulted in similar differences in engraftment of wt and Robo4 hosts. *p<0.03; **p<0.008; ***p<0.0001.

FIG. 14 shows vascular permeability is increased in Robo4^{-/-} mice. Evans Blue leakage into tissues after Iv infections at steady-state (first three panels) and upon irradiation (BM) supports previous reports that Robo4 stabilizes the vasculature. Evans Blue was extracted from tissue within 10 minutes of injection and measured by spectrophotometry. For BM experiments, Evans Blue was injected 24 hrs post-lethal irradiation Data represent three to five independent experiments. *p<0.04; **p<0.002; ***p<0.001.

FIG. 15 shows VEGF injections, alone or in combination with AMD increase the number HSC in PB. Data are from 3 independent experiments. * P<0.05; **p<0.03; ***p<0.001.

FIG. 16A illustrates experimental results of co-mobilization of hematopoietic stem cells (Mob HSC) and multipotent progenitors (Mob MPP) using AMD3100 and rhVEGF-165. Note that bar marked “PBS” should read “HBSS”.

FIG. 16B illustrates experimental results of treatment using rhVEGF-165 (Vegf) and AMD3100 (AMD) to mobilize the granulocyte/macrophage (GM) compartment determined over a period of at least twelve weeks. The results show that, unexpectedly, the combination of rhVEGF-165 and AMD3100 resulted in a more pronounced mobilization of cells compared with simply adding the effects of rhVEGF-165 and AMD3100 separately. This is an unexpectedly superior result.

FIG. 17 illustrates experimental results showing that pretreatment of mice to deplete the bone marrow of stem cells and progenitor cells using either radiation or rhVEGF-165 followed by treatment with AMD3100 results in a chimeric GM population.

FIG. 18 illustrates a time course of co-mobilization of hematopoietic stem cells and multipotent progenitors using AMD3100 and rhVEGF-165. Bar marked “PBS” should read “HBSS”. In particular, note the rapid appearance of HSC and MPP at one hour post-treatment. As before the results show that, unexpectedly, the combination of rhVEGF-165 and AMD3100 resulted in a more pronounced mobilization of cells compared with simply adding the effects of rhVEGF-165 and AMD3100 separately. This was an unexpectedly superior result that could not have been predicted by knowledge of the prior art.

### DETAILED DESCRIPTION OF THE INVENTION

Robo4 Regulates HSC Interactions with Bone Marrow (BM) Niches

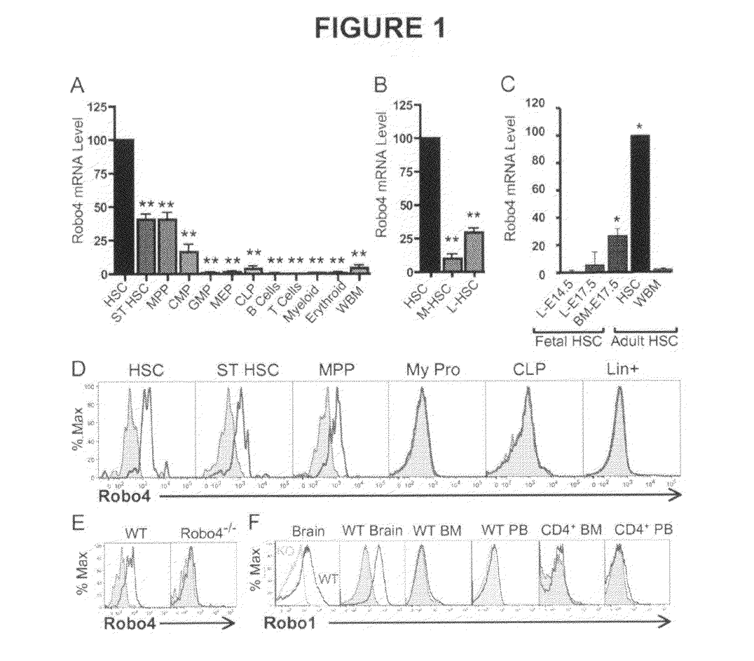

We have identified Robo4 as a critical regulator of HSC localization to the bone marrow (BM). Robo4 expression was very low in fetal HSC residing in the liver, but increased during development concurrent with the establishment of BM hematopoiesis (FIG. 1C). Thus, Robo4 is very selectively expressed by adult BM HSC, and downregulation occurs not only during normal differentiation, but also upon HSC mobilization and in leukemogenesis (FIG. 1A,B). Intriguingly, these processes all involve alterations in cell location, concomitant with a surge in proliferation. Interestingly, L-HSC from the JunB mouse model of myeloproliferative disorder display niche-independent proliferation and aberrant localization to PB, spleen and liver (Passegue et al., 2004). Although we have not yet assessed the functional role of Robo4 in leukemic transformation, its downregulation in L-HSC is consistent with the proposed tumor suppressor functions of Robo receptors (Dallol et al., 2002; Legg et al., 2008; Marlow et al., 2008, Narayan, 2006 #189). Thus, downregulation of Robo4 may be a prerequisite for HSC exit out of BM niches regulating IBC function. As very few BM cells are Robo4-positive, our data suggest that Robo4 is an excellent HSC-specific marker. It will be interesting to investigate the utility of Robo4, alone and in combination with other highly specific HSC markers such as Esam1 (Ooi et al., 2009), in simplified HSC purification protocols.

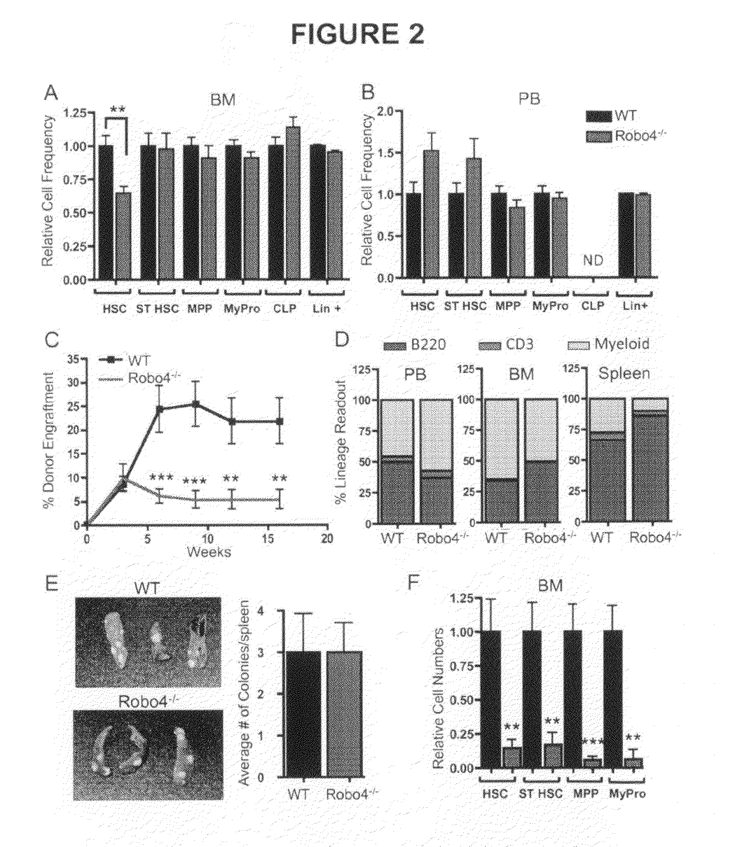

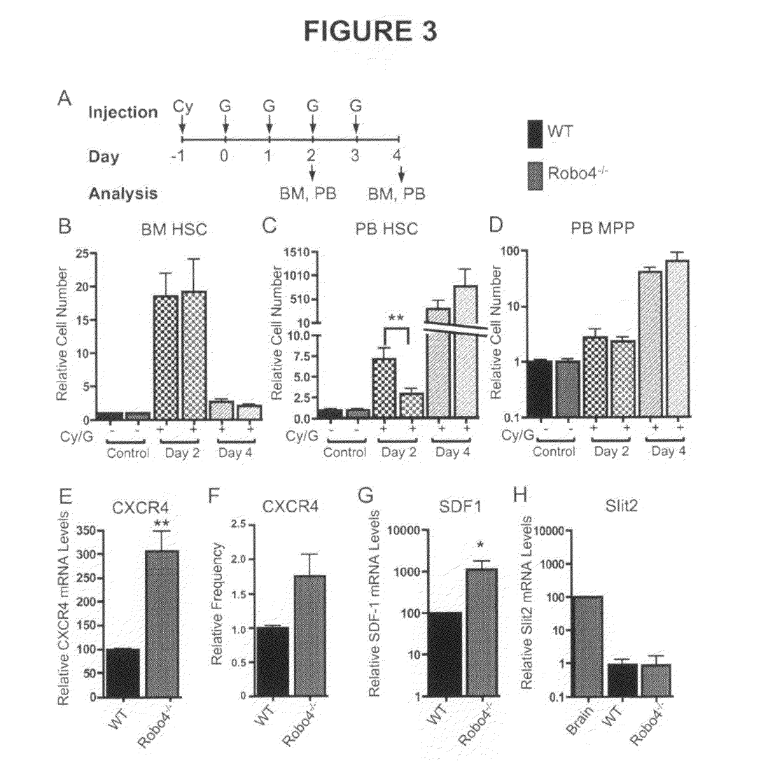

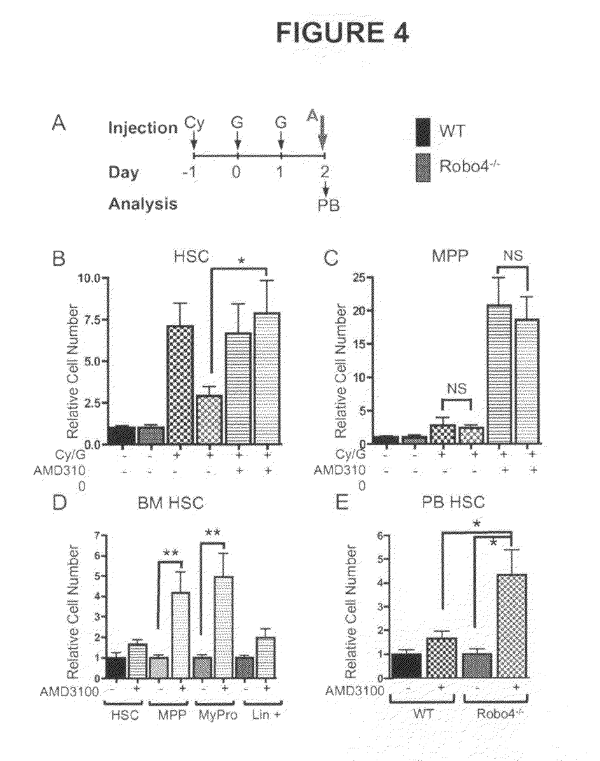

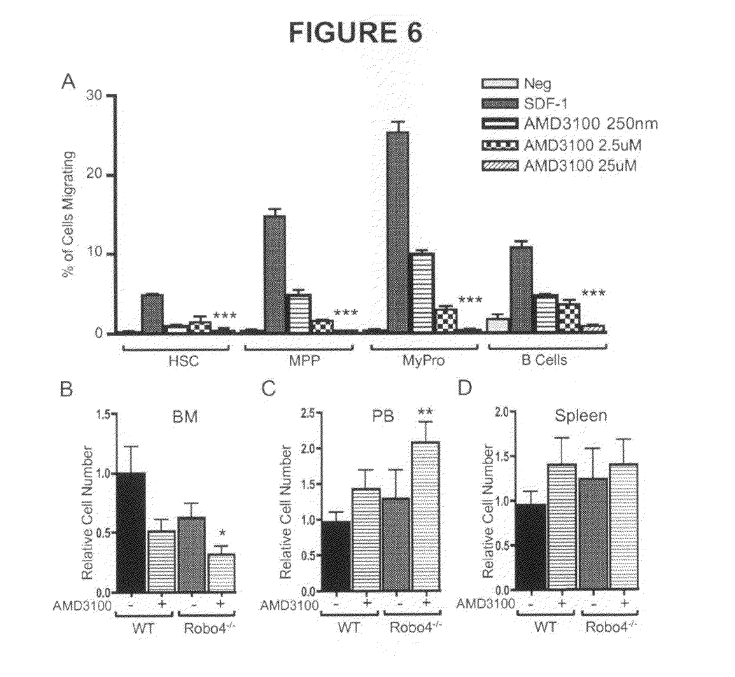

Consistent with its HSC-specific expression, Robo4 deletion led to perturbations in HSC localization during steady-state (FIG. 2A), in short-term homing (FIG. 6) and long-term reconstitution assays (FIG. 2C,F), and upon mobilization with both Cy/G and AMD3100 (FIGS. 3 and 4). These effects were specific for BM localization, as spleen readouts and in vitro HSC properties were not affected by Robo4 loss (FIGS. 2E, 9). Decreased Robo41 HSC frequencies in BM at steady-state indicates that Robo4 stabilizes interactions between HSC and BM niche components. Such a function is consistent with the poor BM localization of Robo4^{-/-} HSC in short-term homing assays, and dramatically impaired long-term engraftment. Importantly, the Robo4^{-/-} HSC that did engraft had normal differentiation capacity (FIG. 2D). Robo4 function therefore appears restricted to regulating HSC interactions with the BM niche, and does not appear to affect cell fate choice. Furthermore, Robo4^{-/-} HSC were more efficiently mobilized with AMD3100 than were wt HSC (FIG. 4E), indicating that Robo4 acts to retain HSC in BM niches. In contrast to the increased relocation to the blood with AMD3100, Cy/G-induced HSC mobilization was impaired in Robo4^{-/-} mice (FIG. 3C). Investigation of the underlying molecular mechanisms revealed that Cxcr4 was upregulated in Robo4^{-/-} HSC (FIG. 3E,F), suggesting that Cxcr4 can compensate for loss of Robo4. Importantly, addition of AMD3100 to the Cy/G regimen restored the mobilization efficiency to wt levels (FIG. 4B). This demonstrates that Cxcr4 and Robo4 act together to retain HSC in the BM. Developmental upregulation of Robo4 and our finding that Robo4 tethers HSC specifically to BM niches provide a tantalizing explanation for how HSC gain Cxcr4-independence once seeded in the BM (Nie et al., 2008).

Slit2 does not Affect HSC Function In Vitro

The role of Slits in Robo4 function has been debated, as high-affinity, direct binding of Slit2 protein to Robo4 protein is not detected (Suchting et al., 2005). However, Robo4 expression endows endothelial cells with migratory responses to Slits (Kaur et al., 2006; Kaur et al., 2008; Park et al., 2003), and Slit2-mediated effects in the vasculature and mammary gland are Robo4 dependent (Jones et al., 2008; London et al.; Marlow et al., 2010). These observations have led to the concept that a coreceptor enhances the affinity of Slit2 for Robo4. Proposed coreceptors include Robo1 (Sheldon et al., 2009) and syndecans (Hu, 2001; Johnson et al., 2004; Steigemann et al., 2004). As Robo1 is not expressed by HSC (FIG. 1F), syndecans are more likely coreceptor candidates in HSC. Indeed, we have previously reported differential regulation of syndecan family members between HSC and progenitor cells (Forsberg et al., 2005). To our knowledge, the functional consequences of this differential expression have not been investigated.

The lack of Slit2 effects on HSC proliferation and migration in vitro does not preclude an important role for Slit2 on HSC function in vivo. Indeed, if Robo4 acts to tether HSC to the BM niche, Slits would be expected to have little impact in solution. Instead, lack of Slit2 effects in vitro supports a role for Slit/Robo signaling in niche-dependent HSC function. Upregulation of Slit2 during hematopoietic stress (Shibata et al., 2009) argues for a physiologically important role of Slit2 in HSC function. The relative importance of this role may be amplified in stress situations, analogous to what has been observed upon challenges to vascular integrity (Jones et al., 2008; London et al.; Marlow et al., 2010). Unfortunately, Slit2^{-/-} mice are not viable (Long et al., 2004) and a Slit2 conditional deletion has not been reported, precluding Slit2-deficient mice as an accessible tool to probe in vivo effects on HSC function.

### Differential Efficacy of Cxcr4 Manipulation on Hematopoietic Stem and Progenitor Cells

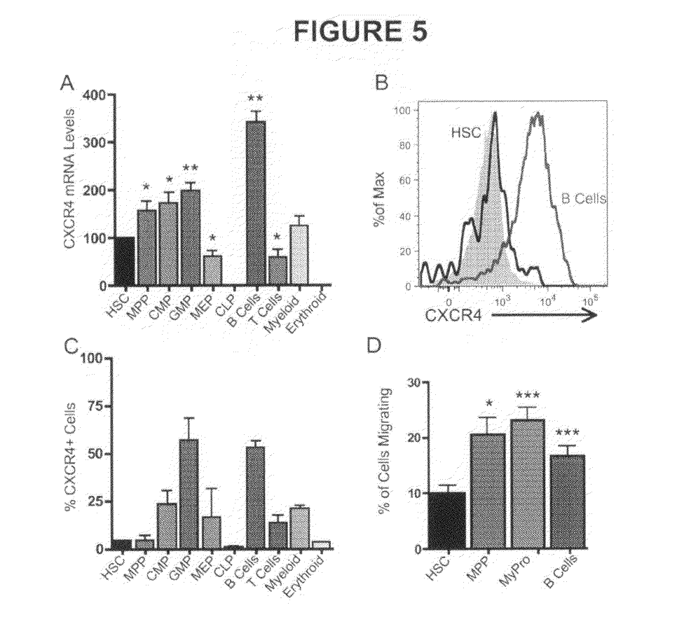

Cxcr4 is a well-established regulator of HSC localization to the BM. Surprisingly, however, we found that HSC express relatively low levels of Cxcr4, both at the transcript and cell surface protein levels. These results contrast those by Sugiyama and colleagues, who reported higher Cxcr4 mRNA levels in HSC compared to MPP (Sugiyama et al., 2006), but are consistent with a recent report assaying Cxcr4 expression and hematopoietic cell migration (Sasaki et al., 2009). Importantly, we showed that differential Cxcr4 expression had functional consequences, as AMD3100-induced mobilization (FIG. 4D) and migration efficiency toward Sdf1 (FIG. 5D) were strictly correlated with Cxcr4 expression levels (FIG. 5). Our findings have important implications for understanding the molecular mechanisms of HSC localization next to Sdf1-expressing cells (Sugiyama et al., 2006). Several cell types, far more numerous than HSC, express higher levels of Cxcr4 (FIG. 5) and consequently respond better to Sdf1 and AMD3100 (FIGS. 4D and 5D). This includes myeloid progenitors, B and T cells. Therefore, molecules other than Cxcr4 must specify location of HSC to limited niche space. Indeed, we show that Robo4 collaborates with Cxcr4 to provide highly HSC-specific localization cues.

Because the molecular mechanisms mobilizing mouse and human HSC are remarkably similar, these findings have potentially important clinical implications. As cells expressing higher levels of Cxcr4 were mobilized more efficiently with AMD3100 (FIGS. 4D and E), PB of AMD3100-mobilized mice contained many more progenitors than longterm reconstituting HSC. This necessitates that quantitation methods are capable of distinguishing progenitors from HSC so as not to overestimate HSC yield. Previous reports analyzing long-term reconstitution capability of PB cells upon AMD3100 mobilization indicated that HSC numbers increase by 1.5-8-fold (Bonig et al., 2009; Broxmeyer et al., 2005), consistent with the ˜1.5-fold increase in PB HSC we observed here (FIG. 4D). These numbers are far lower than those observed with Cy/G treatment, which led to a 300-fold increase in HSC numbers in PB by D4 (FIG. 3C). Because a bolus injection of AMD3100 alone does not yield sufficient numbers of HSC for an adult transplant, alternative injection protocols and combinatorial use with other mobilizing agents have been explored. Such experimentation includes continuous AMD3100 infusion (Bonig et al., 2009), and combinatorial use with G-CSF (Bonig et al., 2009; Flomenberg et al., 2005; Liles et al., 2003) and integrin a4 inhibitors (Bonig et al., 2009). A mobilizing agent specifically targeting HSC, such as an inhibitor of Robo4-mediated adhesion, may significantly boost HSC yield.

### Robo4 and Cxcr4 Employ Distinct Molecular Mechanisms to Localize HSC to the BM

The HSC phenotype upon Robo4 loss is similar to that of conditional deletion or AMD3100mediated inhibition of Cxcr4. For example, deletion of Robo4 and AMD3100 treatment resulted in similar decreases in HSC localization to the BM three hours post-injection (FIG. 6B), and at steady-state, HSC BM frequencies were decreased upon either Robo4 (FIG. 2A) or Cxcr4 deletion (Nie et al., 2008; Sugiyama et al., 2006). In addition, both Robo4^{-/-} and Cxcr4^{-/-} HSC display lower long-term engraftment, but retained lineage multipotency (FIG. 2C, D), (Nie et al., 2008; Sugiyama et al., 2006). However, important differences distinguish the mechanisms of receptor function. Cxcr4 expression endows HSC with an active migratory response toward Sdf1, while we were unable to detect such effects with Slit2. Additionally, Cxcr4 is expressed by many hematopoietic and non-hematopoietic cell types, while Robo4 expression is highly selective for HSC. Indeed, our functional data demonstrate highly HSC-specific functions for Robo4.

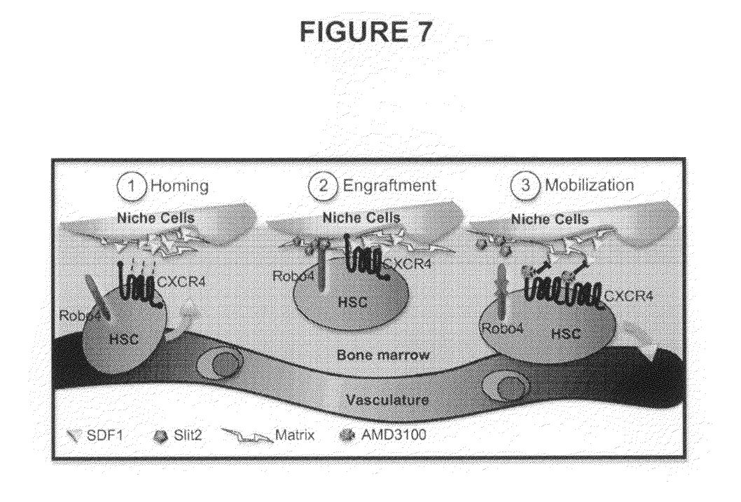

In a simplified model, chemoattractants, including Sdf1, guide HSC to the BM (FIG. 7). Once in the vicinity of HSC-supportive niches, Cxcr4 and Robo4, together, promote HSC retention in the niche and stable engraftment. The highly USC-restricted Robo4 expression likely endows HSC with a competitive advantage to limited BM niche space compared to cells expressing higher levels of Cxcr4, but not Robo4. Inhibition or loss of Cxcr4 results in fewer HSC actively migrating toward niches. Loss of Robo4, on the other hand, likely results in equal, or because of Cxcr4 upregulation maybe even greater, numbers of HSC localizing close to niches. However, BM localization is transient in the absence of Robo4 because fewer HSC engage in stable niche interactions. In both cases, decreased long-term engraftment is observed. Due to these dual cooperative adhesive cues, both Robo4- and Cxcr4-mediated interactions with the niche have to be inhibited for efficient HSC mobilization to the blood; thus, AMD3100-induced HSC mobilization is more efficient in Robo4-deficient mice.

### Receptor Redundancy in the Control of HSC Function

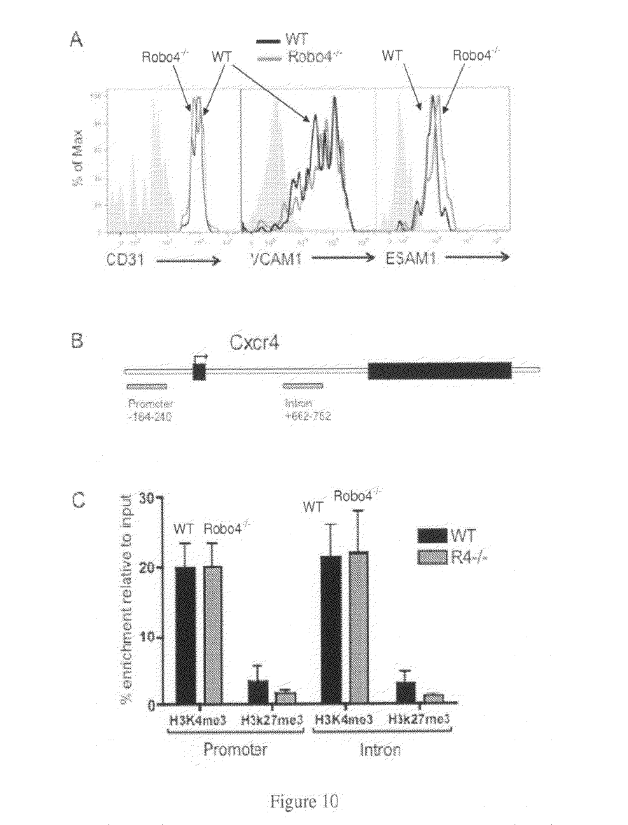

Upregulation of Cxcr4 seems to partially compensate for Robo4 loss and attenuate the phenotype of Robo4^{-/-} mice. This is supported by the inefficient HSC mobilization with Cy/G in Robo4^{-/-} mice (FIG. 3C) and additive effects in BM homing experiments (FIG. 6B). Likewise, engraftment of Cxcr4^{-/-} HSC is likely possible due to functional redundancy with Robo4 and other adhesion receptors expressed by HSC. Although we did not detect upregulation of Vcam1, Esam1 or CD31 upon Robo4 deletion, these receptors are highly expressed by HSC (FIG. 10A), and likely contribute to HSC localization (Kikuta et al., 2000; Ooi et al., 2009; Ross et al., 2008). In the vasculature, Robo4 intersects with pathways regulated by VE-cadherin and VEGF receptors. We recently reported increased defects in angiogenesis under pathological conditions in Robo4^{-/-} mice (Jones et al., 2008) and we also found that Robo4 controls blood vessel growth during mammary gland development (Marlow et al., 2010). These reports demonstrated that Robo4 is dispensable under homeostatic conditions, but critically important during tissue perturbation and remodeling. Mechanistically, it is intriguing that the Sdf1/Cxcr4 axis is upregulated in Robo4^{-/-} mammary glands (Marlow et al., 2008). These results point to conservation of molecular mechanisms across tissues, and between different Robo receptors.

Several molecules have been implicated in HSC homing and engraftment, but the relationship between these factors and how they work together to specify HSC location is unclear. We recently proposed a “niche code hypothesis”, where HSC location is specified by a combination of factors, much like the histone code hypothesis dictates transcriptional outcome (Forsberg and Smith-Berdan, 2009). This model takes into account the contribution of multiple receptors in regulating HSC location and function. Such receptor redundancy would also allow HSC to respond to multiple types of cues to stimulate production of the appropriate cell type. We have begun to dissect this complex regulation by establishing a functional relationship between Robo4 and Cxcr4 in controlling HSC location. A sophisticated understanding of the molecular cues from the endogenous niche milieu that support HSC self-renewal will be necessary to overcome our frustrating inability to expand and generate transplantable HSC ex vivo.

### Therapeutic Potential of Manipulating Robo4 Function

The responsiveness of Robo receptors to soluble ligands renders them optimal targets for manipulation by natural or synthetic agonists and antagonists. A relevant precedence is provided by the clinical utility of Cxcr4 antagonists in hematopoietic cell mobilization. However, Cxcr4 is expressed by many different cell types, including the brain, leading to significant effects on non-HSC populations, and genetic Cxcr4 deletion is embryonic lethal. In contrast, Robo4^{-/-} mice are viable with mild phenotype, and Robo4 expression is restricted to HSC and endothelial cells. Thus, pharmacologic manipulation of Robo4 function will likely be safe and highly specific. HSC-selective mobilization treatments would eliminate the need to treat donors with nonspecific cytotoxic drugs, and improved engraftment would allow reduced myeloablative regimen of the recipient. Mobilization of progenitor cells in the donor and inclusion of these cells in transplants is beneficial for recipients, as progenitors provide rapid regeneration of vital cells. However, when attempting to vacate stem cell niches in recipients to optimize engraftment of transplanted stem cells, very specific targeting of HSC, with retention of progenitor cells, seems ideal for combined stem cell engraftment and minimal compromise of patient health. Moreover, coaxing Robo4 expression on ESC-derived or in vitro expanded HSC may increase the engraftment efficiency of these cells and make such sources of HSC a clinically viable option. Thus, once potent modulators of Robo4 function have been identified, Robo4 is a potentially valuable clinical target.

### Interaction of Robo4 and VEGF

We have demonstrated that the guidance receptor Robo4, by its highly selective expression on HSC, is necessary for robust HSC engraftment. Here, we present data demonstrating that Robo4 expressed by the vascular endothelium, in addition to Robo4 on HSC, promotes HSC engraftment. This discovery was surprising, because Robo4 prevents vascular permeability that is thought to facilitate HSC translocation across vessel walls and subsequent engraftment. We hypothesized that HSC engraftment is regulated by a balance of vascular permeability that facilitates HSC translocation across vessels and vascular integrity that actively promotes HSC extravasation and stable maintenance in BM niches. This hypothesis provides a novel conceptual framework for manipulating the vasculature to affect HSC location and function. We define the spatial and mechanistic requirements for the vascular endothelium in HSC engraftment. The molecular focus is on Robo4, because Robo4 expression by both HSC and vascular endothelial cells (VEC) is necessary for optimal HSC engraftment. Furthermore, Robo4 intersects with VEGF signaling, a master regulator of vascular function. Our ability to manipulate vascular permeability via VEGF and Robo4 provides novel, powerful tools for pursuing the mechanisms of endothelial-mediated promotion of HSC engraftment. The goal of our research is to provide a better understanding of the cellular and molecular mechanisms regulating HSC long-term engraftment upon transplantation. Hematopoietic cell transplantation can provide a complete and permanent cure for a wide range of human disorders. Still, the ˜30% mortality rate of hematopoietic transplant recipients is unacceptably high and transplants are therefore reserved for patients with few other treatment options. By defining the mechanisms regulating HSC homing, extravasation, and retention within BM niches, our goal is to improve HSC long-term reconstitution efficiency.

We have discovered that the integrity of the recipient vasculature influences HSC engraftment efficiency. This is important because it will provide novel strategies for improving hematopoietic transplantation therapies. Based on our new observation that the molecular integrity of vascular barriers affects HSC translocation across vessel wall, we will test whether manipulation of vascular integrity can be used to enhance HSC engraftment and HSC mobilization. Improving HSC mobilization efficiency will increase the numbers of HSC available for transplantation, an issue of particular concern for patients that mobilize poorly due to prior chemotherapy treatments. Likewise, increased engraftment may allow a reduction in myelosuppressive preconditioning of patients. We also test whether Robo4-mediated stabilization of the vasculature mitigates radiation-induced damage of HSC niches in the BM. In addition to reducing the side effects of myelosuppressive conditioning, our investigation of the BM environment will help resolve the controversies regarding HSC niches. This is important, because, despite the undisputed importance of BM niches in supporting HSC function during development, homeostasis, and upon transplantation, the interplay of the cellular and molecular mechanisms responsible for optimal HSC function, including self-renewal, are under intense debate.

Robo4 is a powerful molecular tool for understanding HSC engraftment. Throughout the proposal, the molecular focus is placed on Robo4, a cell surface receptor selectively expressed by HSC and vascular endothelial cells (VEC). The mild phenotype of Robo4^{-/-} mice makes it clear that Robo4 is not necessary for the everyday function of either HSC or VEC. Instead, Robo4 plays important roles in response to perturbations of both the hematopoietic and vascular systems. Because Robo4^{-/-} mice are viable, and most responses are dose-dependent rather than all-or-none, Robo4 provides a quantitative molecular tool for understanding the mechanisms regulating HSC engraftment. Importantly, the partial response to Robo4 manipulation has already prompted us to consider mechanistic and molecular redundancy. Many cell surface receptors that have been implicated in HSC function are, like Robo4, shared between HSC and endothelial cells. These receptors represent a molecular gold mine for manipulating HSC location from both hematopoietic and vascular perspectives.

Understanding HSC trafficking will inform mechanisms of tumor metastasis. The process of cancer metastasis and establishment of secondary tumors is strikingly similar to HSC mobilization, entry into vasculature, trafficking in the blood stream, extravasation, and engraftment in the BM. By defining the role of the vasculature, and molecular mediators of vascular stability (Slit2/Robo4) and permeability (VEGF), in regulating cellular entry and exit into the vasculature, our findings could guide the use of new and current therapies, such as anti-VEGF antibodies, to reduce the incidence of metastasis. Because secondary tumors are significantly more malignant than primary tumors, strategies to reduce metastasis have excellent potential to improve cancer patient prognosis.

We note that Slit2-mediated activation of Robo4 may be of high significance for vascular syndromes, including ARDS, sepsis, radiation damage and HIV infection.

Long-term engraftment is a unique and defining property of HSC and the basis of stem cell transplantation therapies as a permanent cure for a wide range of hematopoietic disorders. HSC transplantation is routinely performed by IV injection of cells, placing a challenging requirement on HSC to travel through the blood stream, adhere to vessel walls near their target destination, and then extravasate from vessels to enter the BM “cavity” where long-term engraftment can take place. HSC must interact with the vascular endothelium at least once during this process. Not surprisingly, the vascular endothelium and many endothelial-selective receptors have been implicated in HSC function. Recent research has largely focused on HSC interaction with vascular niches within the BM, leaving an incomplete picture of the HSC-endothelial interactions occurring during HSC trafficking in blood vessels. An important question is whether the vasculature enhances or impairs HSC engraftment. HSC engraftment in unmanipulated hosts is near non-existent, while robust engraftment is achieved by irradiation preconditioning. Thus, the vascular damage induced by irradiation may enhance engraftment by facilitating the relocation of cells across vessel walls into the BM space. In contrast, severe vascular damage may destroy the BM niches supporting HSC function, and lead to impaired engraftment. We hypothesized that optimal HSC engraftment depends on a balance between not enough and too much vascular damage. We test the possibility that the vascular endothelium acts both as a barrier that contains HSC within vessels and as an active vehicle for delivering HSC to supportive niches within the BM.

We showed that Robo4 cooperates with Cxcr4 to regulate HSC location and mobilization into and out of BM niches (FIG. 2). Robo4^{-/-} HSC engrafted poorly in wt hosts (FIG. 2), demonstrating that HSC-expressed Robo4 promotes HSC engraftment. We have also shown that the Robo ligand Slit2, a large secreted protein, is expressed by BM stromal cells and that its expression is upregulated upon irradiation-induced stress in a pattern strikingly similar to the Cxcr4 ligand SDF1. Combined with the differential Robo4 expression by HSC during development, differentiation, mobilization and leukemogenesis this suggests an increased importance of HSC-expressed Robo4 under dynamic hematopoietic conditions.

Outside the hematopoietic system, Robo4 is expressed by endothelial cells. As shown by our collaborators and others Robo4 expression in the vascular endothelium protects mice from a range of insults by regulating vascular integrity, and administration of exogenous Slit2 reduces vascular damage by a Robo4-dependent mechanism. It has also been shown that Robo4 attenuates permeability induced by VEGF, originally referred to as “vascular permeability factor” and a master regulator of vascular function. We use Slit2 and VEGF as tools to assess the role of Robo4 and vascular permeability in HSC trafficking and location.

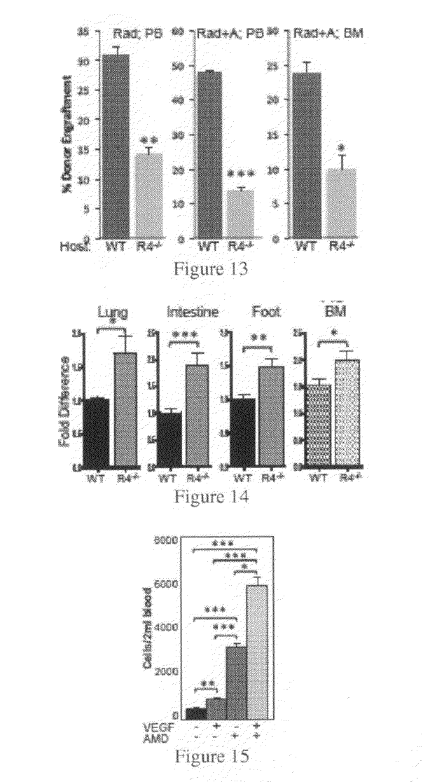

To investigate the mechanisms of Robo4-mediated regulation of HSC function, we transplanted wt HSC into Robo4^{-/-} mice. We found that wt HSC engraft poorly in Robo4^{-/-}-recipients (FIG. 13). This was surprising for two reasons: first, wt HSC have a competitive advantage over Robo4^{-/-} HSC when cotransplanted into wt hosts' and second, we thought that the increased vascular permeability of Robo4^{-/-} mice would facilitate the entry of transplanted cells into the marrow space. Using a modified Miles assay based on Evans Blue leak out of vessels, we have successfully reproduced the reported increased vascular permeability in Robo4^{-/-} mice (FIG. 14). Importantly, we also found that the BM endothelium was hyperpermeable in irradiated Robo4^{-/-} mice (FIG. 14) the conditions under which we discovered differential HSC engraftment (FIG. 13). This means that HSC engraftment was impaired despite increased vascular permeability. Thus, while we had predicted that wt HSC would engraft with high efficiency in Robo4 recipients, instead we found that Robo4 expressed on host cells plays important roles in promoting HSC long-term engraftment. Because the transplanted HSC were wt, this is in addition to, and potentially separate from, the role of HSC-expressed Robo4. We have designed two focused and complementary aims to define the spatial and mechanistic actions of hematopoietic and endothelial Robo4 in supporting HSC engraftment and to test whether manipulation of vascular integrity can be used to improve HSC engraftment and mobilization.

To determine whether vascular permeability affects HSC mobilization to the blood. HSC mobilization in response to both cytoxan/G-CSF (Cy/G) and AMD3100, a CXCR4 inhibitor, is altered in Robo4^{-/-} mice. We concluded that these differences were mediated by Robo4 expressed by HSC, but we did not consider a role for endothelial Robo4. Our new discovery of impaired engraftment of wt HSC in Robo4^{-/-} hosts (FIG. 13) revealed a role for vascular Robo4 in HSC trafficking. We hypothesized that vascular permeability affects the efficiency of HSC mobilization to the blood. Here we test this hypothesis by determining whether Slit2-induced vascular stabilization and VEGF-mediated vascular permeability affects HSC mobilization from BM to PB.

We test whether Slit2-mediated vascular stabilization or VEGF-induced vascular permeability affects AMD-induced hematopoietic mobilization. Wt mice are treated with AMD (administered 1 and 2 hours prior to PB analysis of HSC and progenitor cells, as described previously; see Section G for details) combined with either Slit2 (100 ng/mouse; 6 hrs prior to PB analysis) or VEGF (50 ng/mouse; 20 min prior to PB analysis) HSC and progenitor cells in the PB will be quantified by flow cytometry as described previously, and PB are transplanted to measure the number of functional HSC.

Second, we test whether vascular permeability affects Cy/G-induced HSC mobilization. We follow the standard Cy/G protocol we used to investigate the role of Robo4 deficiency on HSC mobilization, adding Slit2 or VEGF to the regimen as described above. PB and BM will be analyzed as described previously and above. We also test the effects of Slit2 and VEGF on HSC mobilization efficiency in Robo4^{-/-} mice. As Robo4^{-/-} mice have higher vascular permeability than wt mice, they may be hyperpermeable in response to VEGF; however, we do not expect that Slit2 will stabilize the vasculature in Robo4^{-/-} mice.

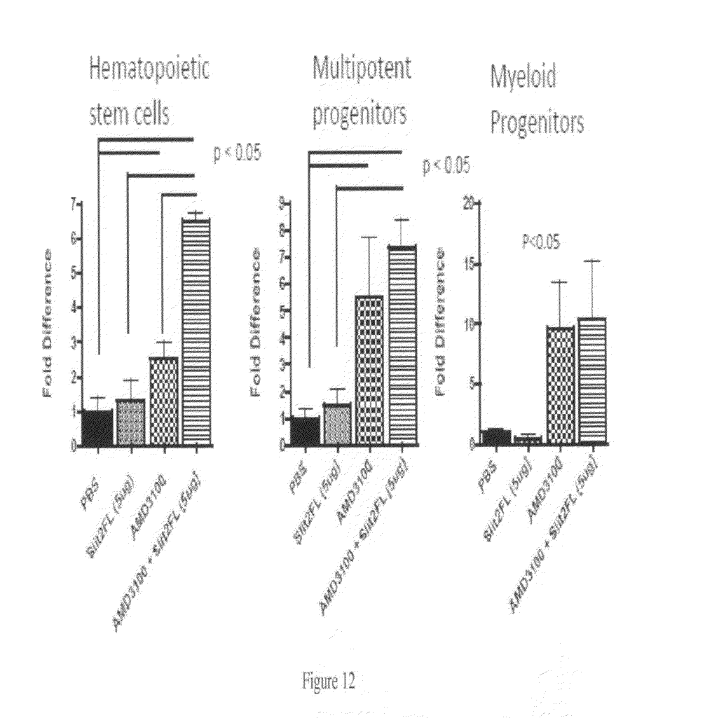

Cy/G and AMD protocols for hematopoietic mobilization are well established and have been successfully implemented by us. Additionally, our results presented in FIG. 12 demonstrate that this aim will lead to exciting new avenues for understanding and improving HSC mobilization. Alternative approaches to alter vascular permeability include the use of mouse models with static, non-drug induced changes in vascular integrity. Because Robo4 has been linked to VE-cadherin function, and VE-cadherin influences leukocyte extravasation, we are particularly interested in testing HSC trafficking in VE-cadherin-catenin fusion transgenic mice.

In one embodiment, the invention provides administering the subject compositions in combination with a pharmaceutically acceptable excipient such as sterile saline or other medium, gelatin, an oil, etc., to form pharmaceutically acceptable compositions. The compositions and/or compounds may be administered alone or in combination with any convenient carrier, diluent, etc., and such administration may be provided in single or multiple dosages. Useful carriers include solid, semi-solid or liquid media including water and non-toxic organic solvents. In another embodiment, the invention provides the subject compounds in the form of a prodrug, which can be metabolically converted to the subject compound by the recipient host. A wide variety of pro-drug formulations for polypeptide-based therapeutics are known in the art. The compositions may be provided in any convenient form including tablets, capsules, troches, powders, sprays, creams, etc. The compositions may further comprise a pharmaceutically acceptable excipient. The compositions may be provided in any convenient form for that may be used to administer the compositions to an individual, for example, as an injectable aqueous or non-aqueous solution or suspension that may be administered to a site of therapy, or administered systematically through an individual's circulatory system. The compositions may be used, for example, in the treatment of a blood disorder, a cancer, metastasis, hematopoietic transplant, etc. The compositions may be used, for example, in stem cell therapy, where cells are administered to an individual for the treatment of a tissue disorder. As such, the compositions, in pharmaceutically acceptable dosage units or in bulk, may be incorporated into a wide variety of containers. For example, dosage units may be included in a variety of containers including capsules, pills, etc. The compositions may be advantageously combined and/or used in combination with other therapeutic or prophylactic agents, different from the subject compounds. The compositions may also be used in in vitro methods for creating stem cell lines, stem cell-derived tissues, and stem cell-derived organs, and the like. In many instances, administration in conjunction with the subject compositions enhances the efficacy of such agents, see, for example, Goodman & Gilman's The Pharmacological Basis of Therapeutics, 9^{th} Ed., 1996, McGraw-Hill.

The invention will be more readily understood by reference to the following examples, which are included merely for purposes of illustration of certain aspects and embodiments of the present invention and not as limitations.

### EXAMPLES

### Example I

### Mice

Mice were maintained by the UCSC animal facility according to approved protocols. Robo4^{-/-} mice were described previously (Jones et al., 2008; London et al.; Marlow et al., 2010). Wt mice were generated from het/het breeding of the Robo4^{-/-} mice or purchased C57BI6 mice from JAX (Bar Harbor, Me.). Radiation was delivered as a split dose administered 3 hours apart using a Faxitron CP-160 X-ray instrument (Lincolnshire, Ill.).

### Example II

### Antibodies

Anti-Robo4 purified antibody (R&D Systems; clone 274914), anti-Robot antibody (purified or biotin-conjugated in house) (Developmental Hybridoma Studies Bank, Univ. of Iowa, vRobo1), Cxcr4-biotin (BD Biosciences); CD31-APC, Vcam1-biotin (clone #429, eBioscience), Esam1 A488 (Nasdala et al., 2002; Ooi et al., 2009) were used in standard protocols with appropriate isotype controls. Other antibodies were described previously (Forsberg et al., 2005; Forsberg et al., 2006).

### Example III

### Cell Isolation and Analysis

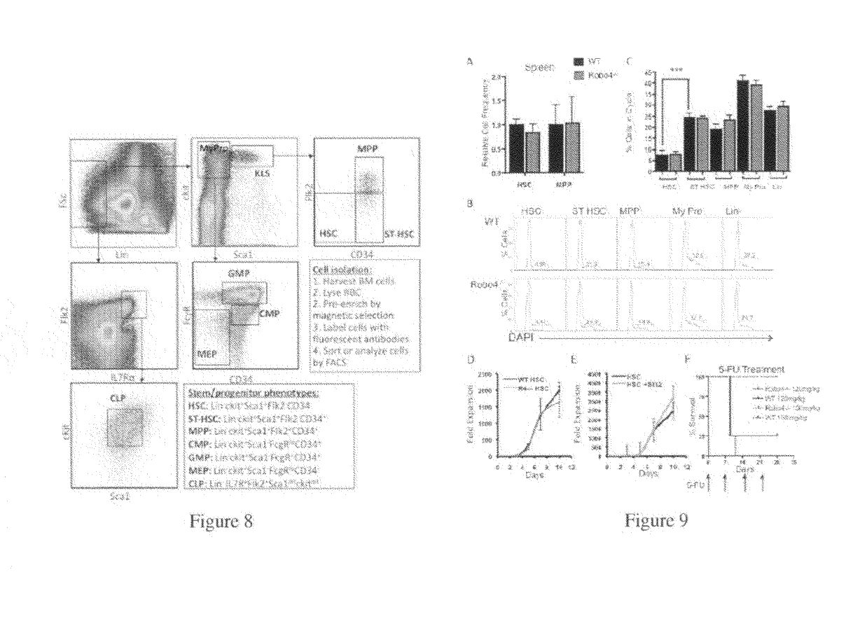

BM, spleen and PB cells were isolated and processed as described previously (Forsberg et al., 2005; Forsberg et al., 2006) using a 4-laser FACSAria or LSRII (BD Biosciences, San Jose, Calif.). Flowjo Software (Ashland, Oreg.) was used for data analysis and display. Unless otherwise indicated, cell populations were defined by the cell surface phenotypes of FIG. 8.

### Example IV

### Competitive Reconstitution Assays

HSC were isolated from Robo4^{-/-} (Ly5.1) or wt (Ly5.1/5.2) donors by two rounds of FACS and administered IV with whole bone marrow helper cells (3e5 cells) from Ly5.2 congenic hosts. Recipient mice were bled at 3, 6, 9, 12 and 16 weeks post transplant via the tail vein and peripheral blood was analyzed for donor chimerism using antibodies to the Ly5.1 (Alexa488) and Ly5.2 (Alexa680) alleles and the lineage markers B220 (APC-Cy7), CD3 (PE), Mac1 (PECy7), Ter119 (PECy5), and Gr1 (Pacific Blue) (eBioscience, Biolegend, or BD Biosciences). Statistically significant differences for all comparisons were calculated using two-tailed t-tests, unless stated otherwise.

### Example V

### qRT-PCR

Quantitative RT-PCR was performed as described previously (Forsberg et al., 2005; Forsberg et al., 2006), except reactions were conducted on a Corbett cycler using the Quantace SensiMixPlus SYBR. Expression of 13-actin was used to normalize cDNA amounts between samples.

### Example VI

### Modified Boyden Migration Assays

BM cells (lineage depleted by magnetic selection, when appropriate), were preincubated at 37° C. for one hour, then placed in the upper chamber of a transwell insert (5 μm pore size). Bottom and/or top wells contained Sdf1 (100 ng/ml) and/or Slit2, as indicated. Cells were allowed to migrate for 2 hrs at 37° C. before harvesting and analysis by flow cytometry.

### Example VII

### Cy/G and AMD3100 Mobilization

Mice were mobilized with cytoxan and G-CSF (Cy/G) as previously described (Morrison et al., 1997). Briefly, mice were injected IP with 200 mg/kg of Cytoxan in HBSS (Sigma-Aldrich) on day −1, followed by 2 or 4 sequential daily sub-cutaneous injections of 200 mg/kg rhG-CSF (Humanzyme, Chicago, Ill.). Tissues were analyzed on day 2 or 4, as indicated (FIGS. 3A, 4A). A cohort from each group was injected IV with 5 mg/kg of AMD3100 1 hour prior to sacrifice. For AMD3100 alone, mice were treated with two serial AMD3100 (5 mg/kg) IV injections one hour apart. Peripheral blood, spleen, and bone marrow were isolated one hour later and processed for cell counts and flow cytometry analysis to determine the numbers and frequencies of each cell population.

### Example VIII

### BM Homing Assays

BM cells were labeled with CFSE labeling dye (Invitrogen) for 5 minutes at ambient temperature, followed by antibody labeling and isolation of cKit⁺/Lin⁻/Sca1⁺/CFSE^{hi} cells by two rounds of FACS. Sorted cells were split in two equal parts and incubated with or without AMD3100 (10 pg/ml) on ice for 30 minutes. Cells were washed, pelleted by centrifugation, and resuspended in HBSS at 400,000 cells/ml. Hosts, lethally irradiated 24 hours prior to transplantation, were injected IV with 40,000 cells in 100 μI. Three hours post-transplant, tissues were harvested from individual mice and analyzed for CFSE labeled cells by flow cytometry. Total cell numbers were counted using a Beckman Coulter Cell Counter.

### Example IX

### Cell Cycle Analysis

Whole bone marrow was isolated from both wt and Robo4^{-/-} and stained for HSC as previously described (Santaguida et al., 2009). Cells were fixed with 2% paraformaldehyde for 5 minutes on ice followed up by a 5 minute permeabilization step using 0.3% Saponin, and 100 units/ml of RNAse A for 30 minutes on ice. 50 uM of DAPI in PBS was added to the cells prior to analysis by flow cytometry.

### Example X

### In Vitro Proliferation

HSC and MPP were isolated and sorted from Robo4^{-/-} and wt mice as previously described. 90-200 cells were plated per well of either strain into a 96-well U-bottom tissue culture plate and cultured for up to 10 days in IMEM medium (Fisher) supplemented with 10% FBS, 25 ng/ml of IL-11, 25 ng/ml of GM-CSF, 25 ng/ml of SCF, 10 ng/ml IL-3 (Cytokines from Peprotech) and Primocin (Invivogen). On days 3, 5, 7, and 10 FITC-labeled spherobeads (BD Bioscience) were added and triplicate samples were analyzed using a FACSAria. Cell expansion rates were calculated based on the number of beads recovered per 10,000 beads added per well.

### Example XI

### Colony Forming Units-Spleen (CFU-S) Assay

100 RISC were transplanted into lethally irradiated C57BI6 hosts anesthetized with inhaled isoflurane (Baxter Pharmaceutical, Deerfield, Ill.) before administration of cells. Cells were delivered with a 27-gauge needle by injection into the posterolateral venous sinus of the orbital cavity. All lethally radiated mice were maintained on water supplemented with antibiotics (106 U/L polymyxin B sulfate and 1.1 g/L neomycin sulfate). 12 days post transplantation recipient mice were euthanized with CO2 inhalation and spleens were surgically removed. Spleens were incubated for 2 minutes in Tellyesniczky's solution (90% ethanol, 5% glacial acetic acid, 5% formalin) prior to scoring the number of macroscopic spleen colonies.

### Example XII

### Chromatin Immunoprecipitation (ChIP)

ChIP protocol was preformed as previously described (Dahl and Collas, 2008). Briefly, 50,000 KLS (cKit⁺/Lin⁻/Sca1⁺) cells were isolated from Robo4^{-/-} and wt mice, cross-linked in 0.5% formaldehyde, lysed and sonicated (Bioruptor, Diagenode). Chromatin was pre-cleared with ProteinA/agarose salmon sperm DNA (Millipore), and incubated with protein A Dynabeads (invitrogen) and 2.5 ug of H3k4me3 and H3k27me3 antibodies (cat#07-473 and 07-449 respectively, Millipore) overnight at 4° C. qPCR for Cxcr4 promoter and intronic regions was performed using the primer locations indicated in FIG. 10B. Results were calculated as percentage (%) of enrichment relative to the amount of input chromatin.

### Example XIII

### qRT-PCR Primers

mRobo4:
Forward:
(SEQ ID NO: 1)
CAGCCTGGTTAGCTCTTCTGATG
Reverse:
(SEQ ID NO: 2)
GCACGAGCAAAGTGAGTATCAGC
Robo1:
Forward:
(SEQ ID NO: 3)
GCTGATTGATTGCCTAACCCTTAAT
Reverse:
(SEQ ID NO: 4)
CAGTGTTGGAAGTCAAGGATAACG
mRobo2:
Forward:
(SEQ ID NO: 5)
CACAGACAGGTGCAGAAGGA
Reverse:
(SEQ ID NO: 6)
CCCGAAGTCTGACGGTACAT
mRobo3:
Forward
(SEQ ID NO: 7)
AAGGATTCCGTGTGTCTTGG
Reverse:
(SEQ ID NO: 8)
GGGCAGTCCTCTTAGCACAG
mCXCR4:
Forward:
(SEQ ID NO: 9)
AGCCTGTGGATGGTGGTGTTTC
Reverse:
(SEQ ID NO: 10)
CCTTGCTTGATGACTCCAAAAG
mSlit2:
Forward:
(SEQ ID NO: 11)
CTGACATGGGACTGTACGCTCATAT
Reverse:
(SEQ ID NO: 12)
GATGTGTCCTTGGGAACTGATGTG
mSDF-1:
Forward:
(SEQ ID NO: 13)
GCCGTTCCTGCTCTCTGCTT
Reverse:
(SEQ ID NO: 14)
ACTCTCCTCCCTTCCATTGCA
mR-actin:
Forward:
(SEQ ID NO: 15)
GACGGCCAGGTCATCACTAT
Reverse:
(SEQ ID NO: 16)
CGGATGTCAACGTCACACTT

### Example XIV

### qPCR Primers for ChIP

Cxcr4 promoter:
Forward:
(SEQ ID NO: 17)
GCGACCCGCAGAAACACCCT
Reverse:
(SEQ ID NO: 18)
GGTCCAGAGCCTGGCCGGATA
Cxcr4 Intron:
Forward:
(SEQ ID NO: 19)
GCTAGTCTGCCGCGTCGCTT
Reverse:
(SEQ ID NO: 20)
AAGCGCTGCTGCTCCATCCC

### Example XV

### Robo4 Expression is Restricted to HSC Tightly Associated with BM Niches

Our previous gene expression microarray analysis showed that Robo4 is expressed at higher levels by HSC compared to MPP, Cy/G-mobilized HSC (M-HSC) and leukemic HSC (L-HSC) (Forsberg et al., 2010; Forsberg et al., 2005). We verified these results by qRT-PCR, and extended the analysis to include multiple BM cell types representing the major hematopoietic progenitor populations and lineages. We found that Robo4 is very selectively expressed by HSC, and downregulated upon differentiation and mobilization, and in leukemogenesis (FIGS. 1A and B). As substantial numbers of M-HSC and L-HSC are found in the blood, spleen and liver (Morrison et al., 1997; Passegue et al., 2004), Robo4 downregulation may facilitate exit from HSC niches in the BM. Intriguingly, Robo4 transcripts were barely detectable in fetal liver HSC and increased significantly in BM HSC during fetal to adult development (FIG. 1C), further emphasizing the specificity of Robo4 expression to HSC located in the BM. Cell surface staining using a monoclonal antibody specific for Robo4 (FIG. 1E) showed that Robo4 protein is robustly expressed by all adult BM HSC, with lower levels on ST-HSC and MPP, and absent from other hematopoietic cell types (FIG. 1D; for flow cytometry gating strategies see Figure Si), in agreement with the qRT-PCR data (FIG. 1A). As <1% of total nucleated BM cells are Robo4-positive, Robo4 is an excellent HSC-specific marker.

Because different Robo receptors may be functionally redundant (Di Meglio et al., 2008; Lopez-Bendito et al., 2007), we also analyzed the expression of Robo1, -2, and -3. Previous studies have reported that circulating hematopoietic cells express Robo1 and respond to the Robo ligand Slit2 (Prasad et al., 2007; Wu et al., 2001). In addition, it has been suggested that Robo4 heterodimerization with Robot is required for Robo4 response to Slits (Sheldon et al., 2009). However, we did not detect robust expression for either Robo1, -2, or -3 in purified hematopoietic cell populations using qRT-PCR under conditions that readily detected these transcripts in brain tissue (data not shown). Additionally, we were unable to detect Robo1 on any BM or PB cell type, including HSC, by flow cytometry using a monoclonal antibody that detected Robo1 on wt, but not Robo4^{-/-}, brain cells (FIG. 1F). These data are consistent with a recent report (Shibata et al., 2009) and suggest that Robo4 is the predominant Robo receptor on hematopoietic cells. Importantly, Robo4 expression is restricted to HSC that maintain tight interactions with the BM niche.

### Example XVI

### Reduced BM Interaction of HSC Lacking Robo4

To assess the functional role of Robo4 in vivo, we analyzed the frequencies of hematopoietic cells in the BM, spleen, and blood of Robo4-deficient mice. Strikingly, analysis of cell frequencies in the BM under normal, non-stress conditions revealed that Robo4^{-/-} mice displayed a significant decrease in HSC frequencies, whereas other cell types were not affected (FIG. 2A). This decrease in HSC BM frequencies was mirrored by a reproducible increase in HSC frequencies in PB (FIG. 2B). HSC numbers in the spleen were not affected (FIG. 9A). To test whether the decrease in HSC BM frequencies reflects defects in HSC proliferation, we assayed proliferative activity in vitro and in vivo. We detected no differences in the cell cycle status of Robo4^{-/-} HSC or progenitors compared to wt mice (FIG. 9B, 9C). We also tested the in vitro expansion rates of wt and Robo4^{-/-} HSC, and whether the putative Robo4 ligand Slit2 elicits a proliferative response on wt HSC, without detecting significant differences (FIG. 9D, 9E). Consistent with these data, Robo4^{-/-} HSC were as able as wt HSC to restore hematopoiesis after weekly injections of the cytotoxic agent 5-fluorouracil (5-FU) (FIG. 9F). Thus, loss of Robo4 does not significantly impair HSC proliferative capacity. Lower HSC frequencies in Robo4^{-/-} BM may instead be explained by reduced HSC retention in the BM. This is supported by the HSC increase in PB in Robo4^{-/-} mice (FIG. 2B) and also by downregulation of Robo4 in M-HSC and L-HSC (FIG. 1B) as mobilization and leukemia lead to higher numbers of HSC in the PB, spleen and liver (Morrison et al., 1997; Passegue et al., 2004).

### Example XVII

### Robo4^{-/-} HSC Display Poor BM Engraftment, but Normal Differentiation Capacity

To test whether Robo4 plays a role in HSC reconstitution of hematopoiesis upon transplantation, we competitively transplanted 100 HSC from wt and Robo4^{-/-} mice into congenic hosts and monitored PB cell readout for 16 weeks. Robo4^{-/-} HSC performed as well as wt HSC up to 3 weeks, but failure to provide sustained hematopoietic expansion over time resulted in a significant difference in PB cell readout beyond 6 weeks (FIG. 2C). The ratios of mature myeloid, B and T cells were not significantly affected by the loss of Robo4 (FIG. 2D). Interestingly, we detected no differences between wt and Robo4^{-/-} HSC in in vivo spleen colony forming assays (CFU-S₁₂) (FIG. 2E), indicating that the impaired transplantation defect is specific for the BM. Indeed, analysis of the BM of long-term reconstituted animals revealed significantly fewer Robo4^{-/-} HSC compared to wt HSC (FIG. 2F). These data show that Robo4^{-/-} HSC display a specific and significantly impaired ability to engraft in the BM. However, the Robo4^{-/-} HSC that do engraft are maintained over time and produce normal ratios of mature cells.

### Example XVIII

### HSC Lacking Robo4 Mobilize Less Efficiently with Cy/G Treatment

Decreased BM frequencies at steady-state (FIG. 2A) and impaired BM engraftment (FIG. 2C, E) of Robo4^{-/-} HSC suggest that Robo4 mediates adhesive interactions between HSC and BM niches. Consequently, Robo4 downregulation upon Cy/G-induced mobilization (FIG. 1B) may be necessary for efficient HSC relocation from BM to PB. We therefore hypothesized that Robo4^{-/-} HSC would be mobilized with greater efficiency compared to wt HSC. To test this directly, we subjected wt and Robo4 mice to the Cy/G injection schedule of FIG. 3A. As expected, wt mice displayed robust increases in BM HSC numbers by day 2 (−20-fold; FIG. 3B), and high numbers of PB HSC starting at D2 with a further significant increase by D4 (−7-fold and −300-fold, respectively, FIG. 3C). Robo4^{-/-} HSC in the BM expanded to similar levels as wt HSC (FIG. 3B), consistent with their normal in vitro proliferation rates and proliferative capacity with in vivo 5-FU treatment (FIG. 9). However, contrary to our hypothesis that Robo4^{-/-} HSC would relocate to the blood more efficiently due to weakened niche interactions, we detected significantly fewer Robo4^{-/-} HSC in the PB at day 2 (FIG. 3C). This impaired mobilization was specific for HSC, as MPP numbers in the PB were similar between wt and Robo4^{-/-} mice at all time points (FIG. 3D).

### Example XIX

### Sdf1 and Cxcr4 are Upregulated to Compensate for Loss of Robo4

To determine whether upregulation of other cell surface receptors accounts for the impaired HSC mobilization in Robo4^{-/-} mice, we compared the expression of potentially redundant receptors in wt and Robo4^{-/-} HSC. We did not detect compensatory increases in Robo1, Robo2 or Robo3 mRNA levels in Robo4^{-/-} HSC (data not shown), and we failed to detect cell surface Robo1 on either wt or Robo4^{-/-} HSC (FIG. 1F and data not shown). Likewise, we detected no differences in the levels of Vcam1, CD31 or Esam1 (FIG. 10A). Because Cxcr4 has been suggested to retain HSC in BM niches by interaction with Sdf1-expressing cells, we assayed the effect of Robo4 deficiency on Cxcr4 expression. Strikingly, we observed a 3-fold increase in Cxcr4 transcript levels in Robo4^{-/-} mice (FIG. 3E). Transcription did not appear to be regulated by levels of histone H3 tri-methylation of lysine 4 (H3K4Me3) and 27 (H3K27Me3) (FIG. 10B, 10C). However, elevated Cxcr4 transcript levels were paralleled by increased cell surface levels of Cxcr4 (FIG. 3F). In addition, we observed an even greater increase in Sdf1 mRNA levels in BM stromal cells of Robo4^{-/-} mice (13-fold, FIG. 3G). Interestingly, expression of Slit2 was not affected by loss of Robo4 (FIG. 3H). These results demonstrate a specific upregulation of the Sdf1/Cxcr4 axis in Robo4^{-/-} BM.

### Example XX

### Inhibition of Cxcr4 Restores Cy/G-Induced HSC Mobilization Efficiency in Robo4^{-/-} Mice

If upregulation of Cxcr4 acts as a compensatory mechanism to counteract the loss of Robo4, inhibition of Cxcr4-mediated interaction with BM niche components should restore mobilization efficiency of Robo4^{-/-} HSC. To test this possibility directly, we performed mobilization assays using Cy/G combined with the Cxcr4 inhibitor AMD3100 according to the injection schedule of FIG. 4A. BM and PB analysis of wt mice revealed no significant differences between treatment with Cy/G alone or Cy/G plus AMD3100 (FIG. 4B). Strikingly, combined Cy/G and AMD3100 treatment of Robo4^{-/-} mice resulted in significantly better HSC mobilization than Cy/G alone (FIG. 4B). Indeed, addition of AMD3100 to the Cy/G regimen restored Robo4^{-/-} HSC levels in the PB to that of wt HSC. This effect was unique to HSC, as there was no differential response between wt and Robo4^{-/-} MPP under these conditions (FIG. 4C). These results support our hypothesis that upregulation of Cxcr4 compensates for loss of Robo4-mediated interactions between HSC and BM niches.

### Example XXI

### Differential Mobilization of Hematopoietic Stem and Progenitors by AMD3100

We also investigated the effects of AMD3100 alone on HSC mobilization in wt and Robo4 mice. While progenitor cell numbers increased robustly in the blood one hour after two sequential AMD3100 injections, we found surprisingly few circulating HSC in wt mice (FIG. 4D). These results were consistent with different injection schedules and routes (IV, SC). Thus, MPP and myeloid progenitors were mobilized more efficiently with AMD3100 than were HSC.

We hypothesized that the relatively low mobilization efficiency with AMD3100 is due to HSC retention in BM niches by non-Cxcr4 mediated, HSC-specific interactions such as Robo4 adhesion. Intriguingly, the efficiency of AMD3100-induced HSC mobilization was much greater in Robo4^{-/-} mice compared to wt mice (FIG. 4E). This supports the hypothesis that Robo4 acts to retain HSC in the BM niche in collaboration with Cxcr4, and that Cxcr4 upregulation compensates for Robo4 loss.

### Example XXII

### HSC Express Relatively Low Levels of Cxcr4 and Migrate Less Efficiently toward Sdf1

When investigating Cxcr4 expression (FIG. 3F), we were surprised to find very low Cxcr4 cell surface levels on wt HSC. Those results and the differential response of HSC and progenitors to AMD3100 (FIG. 5D) prompted us to investigate the relative importance of Cxcr4 for different BM subpopulations. We first compared Cxcr4 expression levels by qRT-PCR. In agreement with published literature, we found very high levels of Cxcr4 transcripts in B cells (FIG. 5A). HSC also expressed Cxcr4 mRNA, although at lower levels than several other cell types. A very similar pattern was observed when analyzing Cxcr4 cell surface levels by flow cytometry (FIG. 5B), revealing that several cell types that are more numerous than HSC display much higher levels of Cxcr4 (FIG. 5C).

We therefore tested the functional consequences of differential Cxcr4 levels by comparing the in vitro migratory response of different populations to Sdf1 (Aiuti et al., 1997). Transwell migration assays revealed a striking correlation between Cxcr4 cell surface levels and migration (FIGS. 5C and D). Although we detected robust and reproducible HSC migration toward Sdf1, cell types expressing higher levels of Cxcr4 (e.g., MPP, myeloid progenitors, and B cells) migrated with significantly greater efficiency. Consistent with this direct correlation, we were unable to detect HSC expression of Cxcr7, the only other proposed Sdf1-responsive receptor (Sierro et al., 2007), using a monoclonal antibody and flow cytometry. These results suggest that the Sdf1/Cxcr4 axis affects hematopoietic progenitor cells to a greater extent than HSC, consistent with the higher mobilization efficiency of progenitors with AMD3100 in vivo (FIGS. 4B, C and D).

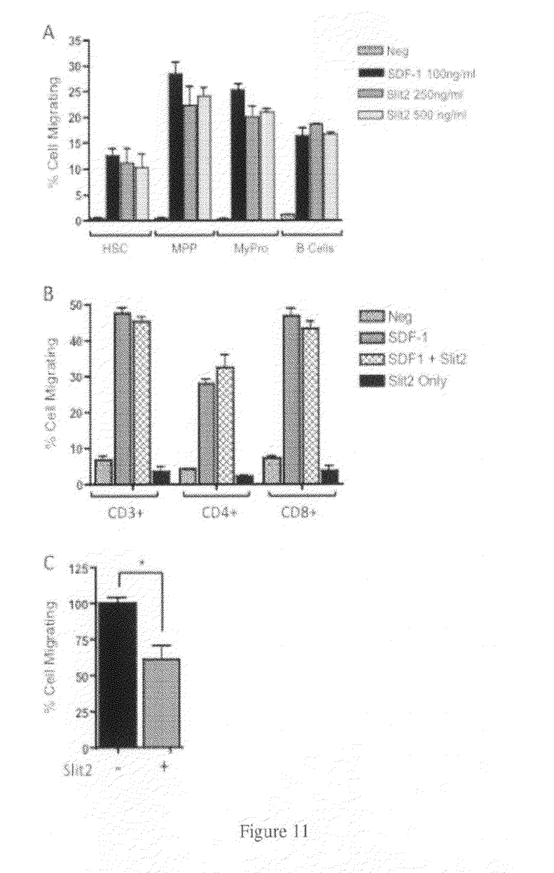

Because Robo receptors on brain and endothelial cells are capable of mediating migratory responses to Slit ligands, we hypothesized that Slit2 might attract or repel HSC. However, we did not detect HSC migration toward Slit2 (data not shown) under conditions where HSC migration toward Sdf1 is readily detected (FIG. 5D). As Slit proteins can act as repellants (Park et al., 2003; Seth et al., 2005), we also tested whether Slit2 inhibited HSC migration toward Sdf1. Neither preincubation of HSC with Slit2 nor addition of Slit2 to Sdf1-containing bottom wells had an effect on Sdf1-induced HSC migration (FIG. 11A); likewise, migration of CD4+T cells was not affected (FIG. 11B). We confirmed that Slit2 was biologically active by demonstrating inhibition of HL60 cell migration toward fMLP (FIG. 11C). Thus, Robo4 expression on HSC does not translate to detectable migratory responses in vitro.

### Example XXIII

### Robo4 and Cxcr4 Cooperate to Localize HSC to the BM Upon Transplantation

The upregulation of Cxcr4 upon loss of Robo4 (FIG. 3E,F), and the increased mobilization efficiency with AMD3100 in Robo4^{-/-} mice (FIG. 4E) prompted us to investigate the role of Cxcr4 and Robo4 on HSC localization to the BM upon transplantation. We first tested whether preincubation with AMD3100 was capable of inhibiting HSC migration toward Sdf1 in transwell migration assays. Indeed, we detected a dose-dependent decrease in cell migration, with complete inhibition at 25 μM of AMD3100 (FIG. 6A).

We then transplanted untreated and AMD3100-treated HSC from wt and Robo4⁻¹ mice into lethally irradiated recipients. Three hours post-injection, BM, spleen, and PB were analyzed for numbers of donor cells. In contrast to in vitro migration, where AMD3100 completely abolished migration of HSC toward Sdf1 (FIG. 6A), AMD3100 was not expected to completely inhibit homing in vivo because Cxcr4^{-/-} HSC are capable of BM engraftment (Nie et al., 2008; Sugiyama et al., 2006). Consistent with this observation, AMD3100 preincubation of wt cells resulted in a ˜2-fold reduction in donor cells localizing to the BM (FIG. 6B). Loss of Robo4 led to a comparable decrease in transplanted cells in the BM (FIG. 6B), a notable result because this decrease occurred despite the elevated levels of Cxcr4 on Robo4^{-/-} HSC (FIG. 3F).

Strikingly, treatment of Robo4-deficient cells with AMD3100 resulted in a further decrease in BM localization (FIG. 6B), demonstrating that both Robo4 and Cxcr4 function to localize HSC to the BM upon transplantation. Consistent with the decreased number of transplanted cells in the BM for each condition, a reciprocal increase of donor cells was detected in the blood stream (FIG. 6C). Interestingly, there were no differences in localization to the spleen (FIG. 6D), supporting the BM-specific effects observed with Robo4^{-/-} HSC in steady state, CFU-S and multilineage reconstitution assays (FIGS. 2 and 9). These data demonstrate that Robo4 and Cxcr4, individually and together, regulate HSC localization to the BM.

BM for each condition, a reciprocal increase of donor cells was detected in the blood stream (FIG. 6C). Interestingly, there were no differences in localization to the spleen (FIG. 6D), supporting the BM-specific effects observed with Robo4^{-/-} HSC in steady state, CFU-S and multilineage reconstitution assays (FIGS. 2 and 9). These data demonstrate that Robo4 and Cxcr4, individually and together, regulate HSC localization to the BM.

### Example XXIV

### Co-Mobilization of Hematopoietic Stem Cells and Multipotenet Progenitors with AMD3100 and rhVEGF-165

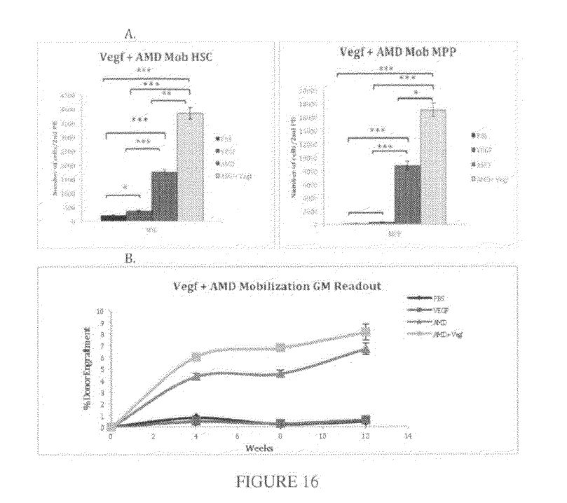

FIG. 16A. 8-16 week old C57B1/6 mice were injected IV with either HBSS, AMD3100 (5 mg/kg SQ), rhVEGF-165 (2 ug/mouse equates to about 0.1 mg/kg), or AMD3100+rhVEGF-165. Mice either received: two doses of HBSS; single dose of HBSS followed 40 minutes later by a single dose of rhVEGF-165; double dose of AMD3100 separated by one hour; or double dose of AMD3100 separated by one hour followed 40 minutes later by a single dose of rhVEGF-165 IV injection. Mice were euthanized by CO2 inhalation 20 minutes after VEGF or 1 hour after AMD3100 injections. The whole peripheral blood from each mouse was individually collected via whole body perfusion with 20 mM EDTA. RBC and WBC suspension was treated with ACK to remove the RBC. Single cell suspensions were individually stained with the appropriate antibody cocktail solution to recognize HSC and other cell types. All antibody incubations were performed on ice for 20-45 minutes with an appropriate concentration of antibody. We analyzed the different cell populations using a 3-laser FACSAria (BD Biosciences, San Jose, Calif.). Flowjo Software (Ashland Oreg.) was used to analyze the expression profiles of the various subsets. Following analysis, the blood samples from each cohort were pooled and injected into sub-lethally irradiated (525 rads single dose administered by a Faxitron CLP-160 X-ray irradiator) congenic hosts; equivalent number of mice injected as were mobilized per cohort.

FIG. 16B. Mice were monitored for blood donor chimerism for 4 months. As before, all antibody incubations were performed on ice for 20-45 minutes with an appropriate concentration of antibody. We analyzed the different cell populations using a 4-laser FACSAria (BD Biosciences, San Jose, Calif.). Flowjo Software (Ashland Oreg.) was used to analyze the expression profiles of the various subsets. Data represent 3 individual experiments, n=3-5 mice per cohort. (*p<0.5, **p<0.05, ***p<0.001.)

### Example XXV

### rhVEGF-165 and Radiation Pre-Treatment Effectively Vacates the Bone Marrow Niche of Stem and Progenitor Cells, Resulting in Increased Long Term Engraftment of Donor Cells Above Radiation Pre-Treatment Only

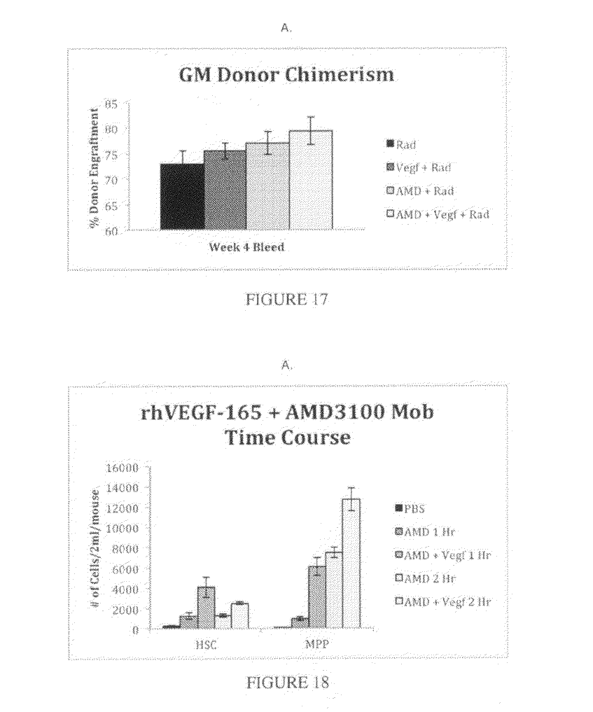

FIG. 17A. 8-16 week old C57B1/6 mice were injected IV with either HBSS, AMD3100 (5 mg/kg SQ), rhVEGF-165 (2 ug/mouse equates to about 0.1 mg/kg), or AMD3100+rhVEGF-165 (at doses listed). Mice either received: two doses of HBSS prior to radiation; single dose of HBSS followed by single dose of rhVEGF-165 20 minutes prior to radiation; double dose of AMD3100 separated by one hour prior to radiation; or double dose of AMD3100 separated by one hour followed by rhVEGF-165 IV injection 20 minutes prior radiation (all mice were dosed with a single dose of 525 rads). Whole bone marrow was isolated from congenic donors (8-16 week old BoyJ) briefly; the femurs and tibias from each mouse were excised. Single cell suspension from the BM was generated by crushing the bones with a mortar and pestle, red blood cells were lysed during a 5-minute incubation in 0.15 M ammonium chloride and 0.01 M potassium bicarbonate solution on ice, and finally the cell suspension was filtered through a 70 micron nylon mesh. Mice were monitored for blood donor chimerism for 4 months. As before, all antibody incubations were performed on ice for 20-45 minutes with an appropriate concentration of antibody. We analyzed the different cell populations using a 4-laser FACSAria (BD Biosciences, San Jose, Calif.). Flowjo Software (Ashland Oreg.) was used to analyze the expression profiles of the various subsets. Data represent 3 individual experiments, n=3-5 mice per cohort. Preliminary data suggests a trend of increased engraftment in the granulocyte/macrophage (GM) compartment, however the differences are not significant at 4 weeks with the current regimen.

### Example XXVI

### Co-Mobilization of Hematopoietic Stem Cells and Multipotenet Progenitors with AMD3100 and rhVEGF-165 More Efficiently Under One Hour Dosing Regimen

FIG. 18A. 8-16 week old C57BIa mice were injected IV with either HBSS, AMD3100 (5 mg/kg SQ), rhVEGF-165 (2 ug/mouse equates to about 0.1 mg/kg), or AMD3100+rhVEGF-165 (at doses listed). Mice either received: two doses of HBSS; single dose of HBSS followed by single dose of rhVEGF-165 20 minutes prior to euthanization later; double dose of AMD3100 separated by one hour; or double dose of AMD3100 separated by one hour followed by rhVEGF-165 IV injection 20 minutes prior to euthanization. Mice were allowed to rest for 1 hour, followed by euthanized by CO2 inhalation. The whole peripheral blood from each mouse was individually collected via whole body perfusion with 20 mM EDTA. RBC and WBC suspension was treated with ACK to remove the RBC. The single cell suspension was individually stained with the appropriate antibody cocktail solution contingent on the use of the cells. All antibody incubations were performed on ice for 20-45 minutes with an appropriate concentration of antibody. We analyzed the different cell populations using a 3-laser FACSAria (BD Biosciences, San Jose, Calif.). Flowjo Software (Ashland Oreg.) was used to analyze the expression profiles of the various subsets. (This data represents a pilot experiment with an n=2 per cohort.)

### REFERENCES

Aiuti, A., Webb, I. J., Bleul, C., Springer, T., and Gutierrez-Ramos, J. C. (1997). The chemokine SDF-1 is a chemoattractant for human CD34+ hematopoietic progenitor cells and provides a new mechanism to explain the mobilization of CD34+ progenitors to peripheral blood. J Exp Med 185, 111-120.
Bonig, H., Chudziak, D., Priestley, G., and Papayannopoulou, T. (2009). Insights into the biology of mobilized hematopoietic stem/progenitor cells through innovative treatment schedules of the CXCR4 antagonist AMD3100. Exp Hematol 37, 402-415 e401.
Broxmeyer, H. E., Orschell, C. M., Clapp, D. W., Hangoc, G., Cooper, S., Plett, P. A., Liles, W. C., Li, X., Graham-Evans, B., Campbell, T. B., Calandra, G., Bridger, G., Dale, D. C., and Srour, E. F. (2005). Rapid mobilization of murine and human hematopoietic stem and progenitor cells with AMD3100, a CXCR4 antagonist. J Exp Med 201, 1307-1318.
Dahl, J. A. and Collas, P. (2008) A rapid micro chromatin immunoprecipitation assay (microChiP). Nat Protoc. 3 (6), 1032-45.
Dallol, A., Forgacs, E., Martinez, A., Sekido, Y., Walker, R., Kishida, T., Rabbitts, P., Maher, E. R., Minna, J. D., and Latif, F. (2002). Tumour specific promoter region methylation of the human homologue of the Drosophila Roundabout gene DUTT1 (ROBO1) in human cancers. Oncogene 21, 3020-3028.
Di Meglio, T., Nguyen-Ba-Charvet, K. T., Tessier-Lavigne, M., Sotelo, C., and Chedotal, A. (2008). Molecular mechanisms controlling midline crossing by precerebellar neurons. J Neurosci 28, 6285-6294.
Flomenberg, N., DiPersio, J., and Calandra, G. (2005). Role of CXCR4 chemokine receptor blockade using AMD3100 for mobilization of autologous hematopoietic progenitor cells. Acta Haematol 114, 198-205.
Forsberg, E. C., Passegue, E., Prohaska, S. S., Wagers, A J., Koeva, M., Stuart, J. M., and Weissman, I. L. (2010). Molecular signatures of quiescent, mobilized and leukemia-initiating hematopoietic stem cells. PLoS One 5, e8785.
Forsberg, E. C., Prohaska, S. S., Katzman, S., Heffner, G. C., Stuart, J. M., and Weissman, I. L. (2005). Differential Expression of Novel Potential Regulators in Hematopoietic Stem Cells. PLoS Genet 1, e28.
Forsberg, E. C., Serwold, T., Kogan, S., Weissman, I. L., and Passegue, E. (2006). New evidence supporting megakaryocyte-erythrocyte potential of flk2/flt3+ multipotent hematopoietic progenitors. Cell 126, 415-426.
Forsberg, E. C., and Smith-Berdan, S. (2009). Parsing the niche code: the molecular mechanisms governing hematopoietic stem cell adhesion and differentiation. Haematologica 94, 1477-1481.
Hu, H. (2001). Cell-surface heparan sulfate is involved in the repulsive guidance activities of Slit2 protein. Nat Neurosci 4, 695-701.
Huminiecki, L., Corn, M., Suchting, S., Poulsom, R., and Bicknell, R. (2002). Magic roundabout is a new member of the roundabout receptor family that is endothelial specific and expressed at sites of active angiogenesis. Genomics 79, 547-552.
Johnson, K. G., Ghose, A., Epstein, E., Lincecum, J., O'Connor, M. B., and Van Vactor, D. (2004). Axonal heparan sulfate proteoglycans regulate the distribution and efficiency of the repellent slit during midline axon guidance. Curr Biol 14, 499-504.
Jones, C. A., London, N. R., Chen, H., Park, K. W., Sauvaget, D., Stockton, R. A., Wythe, J. D., Suh, W., Larrieu-Lahargue, F., Mukouyama, Y. S., Lindblom, P., Seth, P., Frias, A., Nishiya, N., Ginsberg, M. H., Gerhardt, H., Zhang, K., and Li, D. Y. (2008). Robo4 stabilizes the vascular network by inhibiting pathologic angiogenesis and endothelial hyperpermeability. Nat Med 14, 448-453.
Kaur, S., Castellone, M. D., Bedell, V. M., Konar, M., Gutkind, J. S., and Ramchandran, R. (2006). Robo4 signaling in endothelial cells implies attraction guidance mechanisms. J Biol Chem 281, 11347-11356.
Kaur, S., Samant, G. V., Pramanik, K., Loscombe, P. W., Pendrak, M. L., Roberts, D. D., and Ramchandran, R. (2008). Silencing of directional migration in roundabout4 knockdown endothelial cells. BMC Cell Biol 9, 61.
Kikuta, T., Shimazaki, C., Ashihara, E., Sudo, Y., Hirai, H., Sumikuma, T., Yamagata, N., Inaba, T., Fujita, N., Kina, T., and Nakagawa, M. (2000). Mobilization of hematopoietic primitive and committed progenitor cells into blood in mice by anti-vascular adhesion molecule-1 antibody alone or in combination with granulocyte colony-stimulating factor. Exp Hematol 28, 311-317.
Lapidot, T. (2001). Mechanism of human stem cell migration and repopulation of NOD/SCID and B2mnull NOD/SCID mice. The role of SDF-1/CXCR4 interactions. Ann N Y Acad Sci 938, 83-95.
Legg, J. A., Herbert, J. M., Clissold, P., and Bicknell, R. (2008). Slits and Roundabouts in cancer, tumour angiogenesis and endothelial cell migration. Angiogenesis 11, 13-21.
Liles, W. C., Broxmeyer, H. E., Rodger, E., Wood, B., Hubel, K., Cooper, S., Hangoc, G., Bridger, G. J., Henson, G. W., Calandra, G., and Dale, D. C. (2003). Mobilization of hematopoietic progenitor cells in healthy volunteers by AMD3100, a CXCR4 antagonist. Blood 102, 2728-2730.
Liu, F., Poursine-Laurent, J., and Link, D. C. (2000). Expression of the G-CSF receptor on hematopoietic progenitor cells is not required for their mobilization by G-CSF. Blood 95, 3025-3031.
London, N. R., Zhu, W., Bozza, F. A., Smith, M. C., Greif, D. M., Sorensen, L. K., Chen, L., Kaminoh, Y., Chan, A. C., Passi, S. F., Day, C. W., Barnard, D. L., Zimmerman, G. A., Krasnow, M. A., and Li, D. Y. (2010). Targeting Robo4-dependent slit signaling to survive the cytokine storm in sepsis and influenza. Sci Transl Med 2, 23ra19.
Long, H., Sabatier, C., Ma, L., Plump, A., Yuan, W., Omitz, D. M., Tamada, A., Murakami, F., Goodman, C. S., and Tessier-Lavigne, M. (2004). Conserved roles for Slit and Robo proteins in midline commissural axon guidance. Neuron 42, 213-223.
Lopez-Bendito, G., Flames, N., Ma, L., Fouquet, C., Di Meglio, T., Chedotal, A., Tessier-Lavigne, M., and Marin, O. (2007). Robot and Robo2 cooperate to control the guidance of major axonal tracts in the mammalian forebrain. J Neurosci 27, 3395-3407.
Marlow, R., Binnewies, M., Sorensen, L. K., Monica, S. D., Strickland, P., Forsberg, E. C., Li, D. Y., and Hinck, L. (2010). Vascular Robo4 Restricts Pro-Angiogenic VEGF Signaling in Breast. Proc Natl Acad Sci USA In press.
Marlow, R., Strickland, P., Lee, J. S., Wu, X., Pebenito, M., Binnewies, M., Le, E. K., Moran, A., Macias, H., Cardiff, R. D., Sukumar, S., and Hinck, L. (2008). SLITs suppress tumor growth in vivo by silencing Sdf1/Cxcr4 within breast epithelium. Cancer Res 68, 7819-7827.
Morrison, S. J., Wright, D. E., and Weissman, I. L. (1997). Cyclophosphamide/granulocyte colony-stimulating factor induces hematopoietic stem cells to proliferate prior to mobilization. Proc Natl Acad Sci USA 94, 1908-1913.
Nagasawa, T., Hirota, S., Tachibana, K., Takakura, N., Nishikawa, S., Kitamura, Y., Yoshida, N., Kikutani, H., and Kishimoto, T. (1996). Defects of B-cell lymphopoiesis and bone-marrow myelopoiesis in mice lacking the CXC chemokine PBSF/SDF-1. Nature 382, 635-638.
Nasdala, I., Wolburg-Buchholz, K., Wolburg, H., Kuhn, A., Ebnet, K., Brachtendorf, G., Samulowitz, U., Kuster, B., Engelhardt, B., Vestweber, D., and Butz, S. (2002). A transmembrane tight junction protein selectively expressed on endothelial cells and platelets. J Biol Chem 277, 16294-16303.
Nie, Y., Han, Y. C., and Zou, Y. R. (2008). CXCR4 is required for the quiescence of primitive hematopoietic cells. J Exp Med 205, 777-783.
Ooi, A. G., Karsunky, H., Majeti, R., Butz, S., Vestweber, D., Ishida, T., Quertermous, T., Weissman, I. L., and Forsberg, E. C. (2009). The adhesion molecule esam1 is a novel hematopoietic stem cell marker. Stem Cells 27, 653-661.
Park, K. W., Morrison, C. M., Sorensen, L. K., Jones, C. A., Rao, Y., Chien, C. B., Wu, J. Y., Umess, L. D., and Li, D. Y. (2003). Robo4 is a vascular-specific receptor that inhibits endothelial migration. Dev Biol 261, 251-267.
Passegue, E., Wagers, A. J., Giuriato, S., Anderson, W. C., and Weissman, I. (2005). Global analysis of proliferation and cell cycle gene expression in the regulation of hematopoietic stem and progenitor cell fates. J Exp Med In press.
Passegue, E., Wagner, E. F., and Weissman, I. L. (2004). JunB deficiency leads to a myeloproliferative disorder arising from hematopoietic stem cells. Cell 119, 431-443.
Peled, A., Petit, I., Kollet, O., Magid, M., Ponomaryov, T., Byk, T., Nagler, A., Ben-Hur, H., Many, A., Shultz, L., Lider, O., Alon, R., Zipori, D., and Lapidot, T. (1999). Dependence of human stem cell engraftment and repopulation of NOD/SCID mice on CXCR4. Science 283, 845-848.
Prasad, A., Qamri, Z., Wu, J., and Ganju, R. K. (2007). Slit-2/Robo-1 modulates the CXCL12/CXCR4-induced chemotaxis of T cells. J Leukoc Biol 82, 465-476.
Ross, E. A., Freeman, S., Zhao, Y., Dhanjal, T. S., Ross, E. J., Lax, S., Ahmed, Z., Hou, T. Z., Kalia, N., Egginton, S., Nash, G., Watson, S. P., Frampton, J., and Buckley, C. D. (2008). A novel role for PECAM-1 (CD31) in regulating haematopoietic progenitor cell compartmentalization between the peripheral blood and bone marrow. PLoS One 3, e2338.
Santaguida, M., Schepers, K., King, B., Sabnis, A. J., Forsberg, E. C., Attema, J. L., Braun, B. S., and Passegue, E. (2009). JunB protects against myeloid malignancies by limiting hematopoietic stem cell proliferation and differentiation without affecting self-renewal. Cancer Cell 15, 341-352.
Sasaki, Y., Matsuoka, Y., Hase, M., Toyohara, T., Murakami, M., Takahashi, M., Nakatsuka, R., Uemura, Y., and Sonoda, Y. (2009). Marginal expression of CXCR4 on c-kit(+)Sca-1(+)Lineage (−) hematopoietic stem/progenitor cells. Int J Hematol 90, 553-560.
Seth, P., Lin, Y., Hanai, J., Shivalingappa, V., Duyao, M. P., and Sukhatme, V. P. (2005). Magic roundabout, a tumor endothelial marker: expression and signaling. Biochem Biophys Res Commun 332, 533-541.
Sheldon, H., Andre, M., Legg, J. A., Heal, P., Herbert, J. M., Sainson, R., Sharma, A. S., Kitajewski, J. K., Heath, V. L., and Bicknell, R. (2009). Active involvement of Robot and Robo4 in filopodia formation and endothelial cell motility mediated via WASP and other actin nucleation-promoting factors. FASEB J 23, 513-522.
Shibata, F., Goto-Koshino, Y., Morikawa, Y., Komori, T., Ito, M., Fukuchi, Y., Houchins, J. P., Tsang, M., Li, D. Y., Kitamura, T., and Nakajima, H. (2009). Roundabout 4 is expressed on hematopoietic stem cells and potentially involved in the niche-mediated regulation of the side population phenotype. Stem Cells 27, 183-190.
Sierro, F., Biben, C., Martinez-Munoz, L., Mellado, M., Ransohoff, R. M., Li, M., Woehl, B., Leung, H., Groom, J., Batten, M., Harvey, R. P., Martinez, A. C., Mackay, C. R., and Mackay, F. (2007). Disrupted cardiac development but normal hematopoiesis in mice deficient in the second CXCL12/SDF-1 receptor, CXCR7. Proc Natl Acad Sci USA 104, 14759-14764.
Steigemann, P., Molitor, A., Fellert, S., Jackie, H., and Vorbruggen, G. (2004). Heparan sulfate proteoglycan syndecan promotes axonal and myotube guidance by slit/robo signaling. Curr Biol 14, 225-230.
Suchting, S., Heal, P., Tahtis, K., Stewart, L. M., and Bicknell, R. (2005). Soluble Robo4 receptor inhibits in vivo angiogenesis and endothelial cell migration. FASEB J 19, 121-123.
Sugiyama, T., Kohara, H., Noda, M., and Nagasawa, T. (2006). Maintenance of the hematopoietic stem cell pool by CXCL12-CXCR4 chemokine signaling in bone marrow stromal cell niches. Immunity 25, 977-988.
Watt, S. M., and Forde, S. P. (2008). The central role of the chemokine receptor, CXCR4, in haemopoietic stem cell transplantation: will CXCR4 antagonists contribute to the treatment of blood disorders? Vox Sang 94, 18-32.
Wright, D. E., Bowman, E. P., Wagers, A. J., Butcher, E. C., and Weissman, I. L. (2002). Hematopoietic stem cells are uniquely selective in their migratory response to chemokines. J Exp Med 195, 1145-1154.
Wright, D. E., Cheshier, S. H., Wagers, A. J., Randall, T. D., Christensen, J. L., and Weissman, I. L. (2001). Cyclophosphamide/granulocyte colony-stimulating factor causes selective mobilization of bone marrow hematopoietic stem cells into the blood after M phase of the cell cycle. Blood 97, 2278-2285.
Wu, J. Y., Feng, L., Park, H. T., Havlioglu, N., Wen, L., Tang, H., Bacon, K. B., Jiang, Z., Zhang, X., and Rao, Y. (2001). The neuronal repellent Slit inhibits leukocyte chemotaxis induced by chemotactic factors. Nature 410, 948-952.
Zou, Y. R., Kottmann, A. H., Kuroda, M., Taniuchi, I., and Littman, D. R. (1998). Function of the chemokine receptor CXCR4 in haematopoiesis and in cerebellar development. Nature 393, 595-599.

Those skilled in the art will appreciate that various adaptations and modifications of the just-described embodiments can be configured without departing from the scope and spirit of the invention. Other suitable techniques and methods known in the art can be applied in numerous specific modalities by one skilled in the art and in light of the description of the present invention described herein. Therefore, it is to be understood that the invention can be practiced other than as specifically described herein. The above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

1. A composition, the composition comprising a Robo4 receptor antagonist and a Cxcr4 antagonist.

2. The composition of claim 1, wherein the Robo4 receptor antagonist is a slit ligand.

3. The composition of claim 2, wherein the slit ligand is selected from the group consisting of slit, slit2, and slit-like2.

4. The composition of claim 1, wherein the Cxcr4 antagonist is selected from the group consisting of AMD3100, stromal cell-derived factor (SDF1) ligand, HIV-1 envelope protein gp120, and a bicyclam derivate.

5. The composition of claim 1, further comprising a CSF receptor agonist.

6. The composition of claim 5, wherein the CSF receptor agonist is selected from the group consisting of G-CSF, myelopoietin, and progenipoietin (ProGP).

7. The composition of claim 1, wherein the composition comprises biological activity, the biological activity comprising mobilization of the hematopoietic stem cells between the bone marrow (BM) and the blood.

8. A pharmaceutical composition, the pharmaceutical composition comprising the composition of claim 9 and a pharmaceutically acceptable excipient.

9. A composition, the composition comprising a vascular permeability inducing factor and a Cxcr4 antagonist.

10. The composition of claim 9 wherein the vascular permeability inducing factor is selected from the group consisting of VEGF-A, VEGF-B, VEGF-C, VEGF-D, VEGF-E, recombinant human vascular endothelial growth factor, rhVEGF-165, placental growth factor-1 (PGF1) and PGF2.

11. The composition of claim 10, wherein the vascular permeability inducing factor is a recombinant human vascular endothelial growth factor, 165 amino acid soluble isoform (rhVEGF-165).

12. The composition of claim 9, wherein the Cxcr4 antagonist is selected from the group consisting of AMD3100, a stromal cell-derived factor (SDF1) ligand, HIV-1 envelope protein gp120, and a bicyclam derivate.

13. The composition of claim 9, further comprising a CSF receptor agonist.

14. The composition of claim 13, wherein the CSF receptor agonist is selected from the group consisting of G-CSF, myelopoietin, and progenipoietin (ProGP).

15. The composition of claim 9, wherein the composition comprises biological activity, the biological activity comprising mobilization of hematopoietic stem cells between bone marrow (BM) and blood.

16. A pharmaceutical composition, the pharmaceutical composition comprising the composition of claim 9 and a pharmaceutically acceptable excipient.

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. A method for improving the rate of successful mobilization of hematopoietic stem cells between bone marrow (BM) and blood, the method comprising the steps of (i) providing the composition of claim 1; (ii) introducing the composition to the vicinity of the bone marrow and hematopoietic stem cells; and (iii) allowing the composition to exert a biological effect, the method resulting in improving the rate of successful mobilization of hematopoietic stem cells between bone marrow (BM) and blood.

30. The method of claim 29, wherein the composition is in combination with a pharmaceutically acceptable excipient.

31. A method for improving the rate of successful mobilization of hematopoietic stem cells between bone marrow (BM) and blood, the method comprising the steps of (i) providing the composition of claim 9; (ii) introducing the composition to the vicinity of the bone marrow and hematopoietic stem cells; and (iii) allowing the composition to exert a biological effect, the method resulting in improving the rate of successful mobilization of hematopoietic stem cells between bone marrow (BM) and blood.

32. The method of claim 31, wherein the composition is in combination with a pharmaceutically acceptable excipient.
